(19) United States
(12) Patent Application Publication
(10) Pub. No.: US 2024/0018493 A1
(43) Pub. Date: Jan. 18, 2024

(54) KNOCK-IN OF LARGE DNA FOR LONG-TERM HIGH GENOMIC EXPRESSION

(71) Applicant: The Board of Trustees of the Leland Stanford Junior University, Stanford, CA (US)

(72) Inventors: Michael G. CHAVEZ, Stanford, CA (US); Lei S. QI, Stanford, CA (US)

(73) Assignee: The Board of Trustees of the Leland Stanford Junior University, Stanford, CA (US)

(21) Appl. No.: 18/251,941

(22) PCT Filed: Nov. 10, 2021
(86) PCT No.: PCT/US2021/072335

§ 371 Date: May 5, 2023

Related U.S. Application Data
(60) Provisional application No. 63/111,846, filed on Nov. 10, 2020.

(51) Int. Cl. C12N 9/22; C12N 15/86; C12N 15/10; A61P 37/04

(52) U.S. Cl. CPC C12N 9/22; C12N 15/86; C12N 15/102; A61P 37/04; C12N 2310/20; C12N 2740/15043

(57) ABSTRACT

The present disclosure provides compositions, systems, and methods for genome editing, efficient knock-in of large DNA fragments, and long-term, stable, high expression of integrated transgenes. Also provided are modified cells, vaccines comprising modified cells, and methods of using such cells to induce an immune response.

### CROSS-REFERENCE TO RELATED APPLICATIONS

This application claims the benefit of U.S. Provisional Application No. 63/111,846, filed Nov. 10, 2020. This provisional application is incorporated by reference herein in its entirety for all purposes.

### BACKGROUND

The gene editing field has advanced rapidly following the rise of CRISPR-Cas technology. However, the field remains faced with three major technical issues: 1) efficient knock-in (KI) of large DNA fragments (e.g., greater than 4,000 nucleotides) into a precise genomic locus; 2) long-term, stable, high expression of desired KI fragments; and 3) KI protocols using good manufacture practice (GMP) compatible reagents and materials. Despite the advances of CRISPR-Cas technology, KI efficiency of large genes remains extremely low. Furthermore, even when genes are knocked in successfully, they are often not expressed highly enough or stably enough. For example, synthetic cells engineered using lentiviral systems or adeno-associated virus (AAV) often do not express transgenes to a high level. Further, genes knocked in using these methods are often targeted for silencing by the cell, decreasing the already low transgene expression over time. Many KI procedures for cell manufacture suffer from high cost related to production of GMP-grade materials (e.g., AAV). Thus, a need exists for gene editing techniques which allow efficient KI of large genes which can be expressed highly and stably for long periods of time.

### BRIEF SUMMARY

This summary is a high-level overview of various aspects of the present disclosure and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present disclosure are discussed below.

In one aspect, provided herein is a donor template comprising: a) a payload comprising a nucleotide sequence; b) one or more homology arms comprising nucleotide sequences, wherein the nucleotide sequences are substantially identical to at least one locus in a genome; and c) one or more cleavage sites comprising nucleotide sequences, wherein the nucleotide sequences can be bound or cleaved by a nuclease.

In some embodiments, the donor template is single-stranded. In some embodiments, the donor template is double-stranded. In some embodiments, the donor template is a plasmid or a DNA fragment or a vector. In some embodiments, the donor template is a plasmid comprising elements necessary for replication, optionally comprising a promoter and a 3′ UTR.

In some embodiments, the donor template is a viral vector. In some embodiments, the viral vector is selected from the group consisting of retroviral, lentiviral, adenoviral, adeno-associated viral, herpes simplex viral, Alphaviral, Rhabdoviral, Newcastle disease viral, Picornaviral, poxviral, Coxsackieviral, and measles viral vectors. In some embodiments, the vector is a modified viral vector selected from the group consisting of retroviral, lentiviral, adenoviral, adeno-associated viral, herpes simplex viral, Alphaviral, flaviviral, Rhabdoviral, Newcastle disease viral, Picornaviral, poxviral, Coxsackieviral, and measles viral vectors. In some embodiments, the vector is a retroviral vector. In some embodiments, the retroviral vector is a lentiviral vector. In some embodiments, the viral vector further comprises genes necessary for replication, transcription, or reverse transcription of the viral vector.

In some embodiments, the donor template or vector comprises one or more homology arms comprising nucleotide sequences, wherein the nucleotide sequences are substantially identical to at least one locus in a genome, wherein the genome is a mammalian genome. In some embodiments, the genome is a human genome.

In some embodiments, the payload of the donor template or vector comprises a nucleotide sequence of at least 4,400 nucleotides. In some embodiments, the payload comprises a nucleotide sequence of at least 4,700 nucleotides. In some embodiments, the payload comprises a nucleotide sequence of at least 6,000 nucleotides. In some embodiments, the payload comprises a nucleotide sequence of up to 4,400 nucleotides. In some embodiments, the payload comprises a nucleotide sequence of up to 4,700 nucleotides. In some embodiments, the payload comprises a nucleotide sequence of up to 8,000 nucleotides. In some embodiments, the payload comprises a nucleotide sequence of up to 8,500 nucleotides.

In some embodiments, the payload of the donor template or vector comprises a transgene. In some embodiments, the transgene does not comprise a promoter. In some embodiments, the transgene comprises a polycistronic expression element. In some embodiments, the polycistronic expression element is selected from the group consisting of: an IRES element, a P2A element, a T2A element, an E2A element, or an F2A element.

In some embodiments, the payload of the donor template or vector comprises a translation enhancement element.

In some embodiments, the one or more homology arms of the donor template or vector independently comprise nucleotide sequences of up to 1,000 nucleotides.

In some embodiments, the one or more cleavage sites of the donor template or vector comprise nucleotide sequences that are substantially identical to a fragment of said at least one locus in the genome.

In some embodiments, the donor template or vector comprises at least two homology arms. In some embodiments, the donor template or vector comprises at least two cleavage sites. In some embodiments, the donor template or vector comprises at least two homology arms and at least two cleavage sites, and the payload, homology arms, and cleavage sites are organized according to the following linear order: cleavage site, homology arm, payload, homology arm, cleavage site.

In some embodiments, the donor template or vector comprises two payloads. In some embodiments, the donor template or vector comprising two payloads comprises at least four homology arms and at least four cleavage sites, and the two payloads, homology arms, and cleavage sites are organized according to the following linear order: cleavage site, homology arm, payload 1, homology arm, cleavage site, cleavage site, homology arm, payload 2, homology arm, cleavage site.

In some embodiments, the donor template or vector comprises more than two payloads (e.g., three payloads, four payloads, five payloads, or more). In some embodiments, each payload is flanked by cleavage sites and homology arms as described above.

In another aspect, provided herein is a system for targeting integration of at least one payload into at least one genomic locus comprising the donor template or vector as described above and a nuclease targeted to the at least one genomic locus. In some embodiments, the genomic locus is in a mammalian genome. In some embodiments, the genomic locus is in a human genome.

In some embodiments, the nuclease of the system is also targeted to the one or more cleavage sites in the donor template or vector. In some embodiments, the nuclease is selected from the group consisting of a CRISPR-associated protein (Cas), a meganuclease, a zinc finger nuclease (ZFN), a transcription activator-like effector nuclease (TALEN), an Argonaute protein, or a transposase.

In some embodiments, the nuclease of the system is a Cas protein and the system further comprises at least one guide nucleic acid to target the Cas protein to the at least one genomic locus. In some embodiments, the Cas protein comprises at least one copy of a nuclear localization signal (NLS). In some embodiments, the Cas protein is Cas9, Cas12, Cas14, a modified version of Cas9, a modified version of Cas12, or a modified version of Cas14.

In some embodiments, the system comprises a vector and the vector is a retroviral vector. In some embodiments, the retroviral vector is a lentiviral vector.

In another aspect, provided herein is a method of targeting integration of at least one payload into at least one genomic locus in a mammalian cell comprising introducing into said mammalian cell at least a first nuclease targeted to the at least one genomic locus and introducing into said mammalian cell a donor template or vector as described above.

In some embodiments, the nuclease of the method is also targeted to the one or more cleavage sites in the donor template or vector. In some embodiments, the nuclease is selected from the group consisting of a CRISPR-associated protein (Cas), a meganuclease, a zinc finger nuclease (ZFN), a transcription activator-like effector nuclease (TALEN), an Argonaute protein, or a transposase.

In some embodiments, the nuclease of the method is a Cas protein and the method further comprises introducing into the mammalian cell at least one guide nucleic acid to target the nuclease to the at least one genomic locus. In some embodiments, the Cas protein comprises at least one copy of a nuclear localization signal (NLS). In some embodiments, the Cas protein is Cas9, Cas12, Cas14, a modified version of Cas9, a modified version of Cas12, or a modified version of Cas14. In some embodiments, introducing the nuclease in the method comprises introducing into the mammalian cell a polypeptide or a nucleic acid encoding said polypeptide, and introducing the at least one guide nucleic acid comprises introducing into the mammalian cell the at least one guide nucleic acid or a nucleic acid encoding said at least one guide nucleic acid.

In some embodiments, the method as described above comprises introducing into the mammalian host cell a vector and the vector is a retroviral vector. In some embodiments, the retroviral vector is a lentiviral vector. In some embodiments, a pseudovirus (e.g., a lentivirus) is used to introduce the lentiviral vector into the mammalian host cell. In some embodiments, the pseudovirus is integration-deficient. In some embodiments, the pseudovirus comprises a mutant integrase protein comprising a D64V substitution.

In some embodiments, the method as described above targets integration of at least one payload into at least one genomic locus in a mammalian cell, wherein the at least one genomic locus comprises a gene with a promoter. In some embodiments, the gene is highly expressed. In some embodiments, the gene encodes a protein that is required for survival of the mammalian cell. In some embodiments, the gene is selected from the group consisting of beta-actin, cytochrome P450, ribosomal subunit S19, IL2 receptor gamma, and CD3 epsilon chain. In some embodiments, the gene is selected from the group consisting of beta-actin and IL2 receptor gamma. In some embodiments, the gene is selected from the group consisting of oncogenes, tumor suppressor genes, and lineage marker genes. In some embodiments, the at least one payload of the method comprises a transgene without a promoter and a polycistronic expression element, and the promoter at the at least one genomic locus can drive expression of the transgene following integration of the payload at said at least one genomic locus. In some embodiments, the promoter at the at least one genomic locus can drive expression of both the gene and the integrated transgene. In some embodiments, the mammalian cell is selected against if it silences transgene expression.

In some embodiments, the method as described above further comprises producing one or more single-stranded breaks at said at least one genomic locus. In some embodiments, the method further comprises producing at least one double-stranded break at said at least one genomic locus. In some embodiments, the at least one genomic locus is modified by homologous recombination using the donor template or vector.

In some embodiments, introducing the donor template or vector in the method as described above occurs at least 12 hours prior to introducing the nuclease. In some embodiments, introducing the donor template or vector occurs at the same time as introducing the nuclease.

In another aspect, provided herein is a pseudovirus comprising the donor template or vector as described above. In some embodiments, the pseudovirus is integration deficient. In some embodiments, the pseudovirus comprises a mutant integrase protein comprising a D64V substitution. In some embodiments, the donor template or vector of the pseudovirus is located between long terminal repeats (LTRs) in the lentiviral genome.

In another aspect, provided herein is a system for targeting integration of at least one payload into at least one genomic locus comprising the pseudovirus as described above and a nuclease targeted to the at least one genomic locus.

In some embodiments, the nuclease of the system is also targeted to the one or more cleavage sites in the donor template or vector of the pseudovirus. In some embodiments, the nuclease is selected from the group consisting of a CRISPR-associated protein (Cas), a meganuclease, a zinc finger nuclease (ZFN), a transcription activator-like effector nuclease (TALEN), an Argonaute protein, or a transposase.

In some embodiments, the nuclease of the system is a Cas protein and the system further comprises at least one guide nucleic acid to target the Cas protein to the at least one genomic locus. In some embodiments, the Cas protein comprises at least one copy of a nuclear localization signal (NLS). In some embodiments, the Cas protein is Cas9, Cas12, Cas14, a modified version of Cas9, a modified version of Cas12, or a modified version of Cas14.

In some embodiments, the pseudovirus of the system comprises a vector and the vector is a retroviral vector. In some embodiments, the retroviral vector is a lentiviral vector.

In another aspect, provided herein is a modified mammalian cell comprising at least one payload integrated into its genome according to any of the methods described above. In some embodiments, the modified mammalian cell is selected from the group consisting of primary human T cells, human dendritic cells, or mouse T cells.

In some embodiments, the modified mammalian cell is a lymphocyte, a phagocytic cell, a granulocytic cell, or a dendritic cell. In some embodiments, the modified mammalian cell is a lymphocyte, and the lymphocyte is a T cell, a B cell, or a natural killer (NK) cell.

In some embodiments, the modified mammalian cell is a T cell, and the T cell is a CD4+ helper T cell or a CD8+ killer T cell. In some embodiments, the modified mammalian cell is a phagocytic cell, and the phagocytic cell is a monocyte or a macrophage. In some embodiments, the modified mammalian cell is a granulocytic cell, and the granulocytic cell is a neutrophil or a mast cell.

In some embodiments, the modified mammalian cell is a stem cell or a progenitor cell. In some embodiments, the modified mammalian cell is a stem cell, and the stem cell is an induced pluripotent stem cell (iPSC), an embryonic stem cell (ESC), an adult stem cell, or a mesenchymal stem cell (MSC). In some embodiments, the modified mammalian cell is a progenitor cell, and the progenitor cell is a neural progenitor cell, a skeletal progenitor cell, a muscle progenitor cell, a fat progenitor cell, a heart progenitor cell, a chondrocyte, or a pancreatic progenitor cell.

In some embodiments, the at least one integrated payload of the modified mammalian cell as described above comprises a transgene expressing an antigen capable of inducing an immune response in a subject. In some embodiments, the antigen is a spike protein from a human coronavirus. In some embodiments, the spike protein is from human SANS-CoV-2. In some embodiments, the antigen is an RNA-dependent RNA polymerase (RdRP) protein from a human coronavirus. In some embodiments, the RdRP protein is from human SARS-CoV-2.

In another aspect, provided herein is a vaccine comprising a modified mammalian cell as described above. In some embodiments, the vaccine further comprises an excipient, an adjuvant, or a combination thereof.

In another aspect, provided herein is a method of inducing an immune response in a subject comprising administering the modified mammalian cell or the vaccine described above. In some embodiments, administering the modified mammalian cell comprises infusing the modified mammalian cell into the subject.

Other objects, features, and advantages of the present disclosure will be apparent to one of skill in the art from the following detailed description and figures.

### BRIEF DESCRIPTION OF THE DRAWINGS

The present disclosure includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows the design of a genome editing system, according to certain aspects of this disclosure. Included are a viral donor template and a nuclease system. In the embodiment shown, the virus is an integrase deficient lentivirus (IDLV), created by a D64V mutation in the viral integrase, and the nuclease system is CRISPR-Cas9. The viral genome comprises a payload comprising a transgene flanked by homology arms that are used for homology directed repair (HDR). The HDR cassette is flanked by cleavage sites that can be cleaved by the nuclease system, freeing it from the viral genetic elements such as long terminal repeats (LTRs).

FIG. 2 shows the mechanism of payload knock-in, according to certain aspects of this disclosure. In the embodiment shown, a retrovirus comprising a donor template is used to infect mammalian cells. Virus infected mammalian cells reverse transcribe the single stranded RNA viral genome into double stranded DNA. Introduction of nuclease to the cell frees the donor template cassette away from viral elements and makes a targeted cut in the genome (upstream of an endogenous gene shown). Homology directed repair knocks in the virally introduced payload at the site of the targeted cut.

FIG. 3 shows integration of a payload upstream of the N-terminal methionine on beta-actin gene (ACTB), according to aspects of this disclosure. The top panel shows the design of an embodiment of a genome editing system with homology arms (HA) that enable integration of GFP directly upstream of ACTB gene. The design is packaged into an IDLV. The CRISPR-Cas9 nuclease system, in the embodiment shown, uses a single guide RNA to cut both the HDR template twice and genomic ACTB (sgACTB). A P2A element is used to separate GFP from ACTB post-translation. The bottom panel shows the result of K562 cells transduced with the IDLV comprising HDR templates with or without the flanking sgACTB cut sites, according to aspects of this disclosure. Some conditions were then electroporated with Cas9-sgACTB ribonucleoprotein (RNP), and cells were analyzed 3, 5, and 7 days post-electroporation via flow cytometry. Cells that had both flanking sgACTB sites and RNP had substantially better knockin efficiencies (indicated by an increase in the proportion of signal shifted right relative to Row 1 [WT cells], representing cells with GFP expression) with less ectopic, non-integrating expression than its counterpart.

FIG. 4 shows that addition of cut sites flanking the HDR cassette improves knock-in efficiency, according to certain aspects of this disclosure. The data shown are from integration of a reporter transgene (green fluorescent protein, or GFP) into the ACTB locus of K562 cells using an integration deficient lentivirus (IDLV) comprising a donor template with or without nuclease cleavage sites and a nuclease and guide RNA system delivered as a ribonucleoprotein (RNP).

FIG. 5 shows that knock-in efficiency is dependent on viral titer and can be predicted using fluorescent intensity at 24 hours, according to certain aspects of this disclosure. The data shown are from K562 cells transduced with GFP IDLV (as shown in FIG. 3, top panel) at various titers. Ectopic, non-integrating expression (as shown in rows 2-4 of FIG. 3, bottom panel) was assayed via flow cytometry 24 hours after transduction, right before electroporation of Cas9-sgACTB RNP. Cells were assayed via flow cytometry 7 days later and knock-in efficiency at day 7 was correlated to GFP median fluorescent intensity (MFI) at 24 hours.

FIG. 6 shows that payloads can be knocked into various genomic locations using the methods of certain aspects of this disclosure. The fluorescent activated cell sorting data shown are from knock-in of a reporter at IL2RG (left panel), ACTB (middle panel), or RAB11A (right panel). The top row of each panel shows reporter signal in wild-type cells, and the bottom row shows reporter signal in knock-in cells.

FIG. 7 shows that large and hard to express genes can be knocked in using the methods of certain aspects of this disclosure. Large transgenes from toxic sources were knocked into the ACTB locus in Jurkat cells and measured by flow cytometry. Transgene A is the toxic S1 region of the SARS-CoV-2 Spike protein and GFP (3.7 kb), Transgene B is the SARS-CoV-2 RNA dependent RNA polymerase (RdRP) and GFP (3.6 kb), Transgene C is the toxic S1, RdRp, and GFP (5.7 kb), and Transgene D is GFP (0.7 kb).

FIG. 8 shows multiple knock-ins from a single viral genome can be made using the methods of certain aspects of this disclosure. The top panel shows a design of a double knock-in strategy where a single IDLV encodes an HDR template that integrates GFP into the N-terminal end of ACTB and mCherry into the N-terminal end of RAB11A. Each template is flanked by its corresponding sgRNA and has a P2A tag to separate the transgene from the endogenous protein. The bottom panel shows results from K562 cells transduced with the IDLV and electroporated with Cas9-RNP complexed with the indicated sgRNA. Cells were assayed via flow cytometry 7 days later.

FIG. 9 shows that knock-ins can be made in therapeutically relevant cell types (primary T cells) using the methods of certain aspects of this disclosure. IDLV containing an HDR template that places GFP-P2A upstream of the N-terminal methionine of ACTB were transduced into human primary I cells and Cas9-sgACTB RNP was electroporated in 24 hours later. Primary human T cells were assayed 7 days later via flow cytometry. The left panel shows a histogram of GFP expression in primary T cells after ACTB knockin. The right panel shows knockin efficiency across three independent donors (Donors A, B, and C).

FIG. 10 shows that knock-ins can be made in therapeutically relevant cell types (primary T cells) using the methods of certain aspects of this disclosure. IDLV containing an HDR template that places GFP-P2A upstream of the N-terminal methionine of IL2RG were transduced into human primary cells and Cas9-sgIL2RG RNP was electroporated in 24 hours later. Primary human T cells were assayed 7 days later via flow cytometry. The left panel shows a histogram of GFP expression in primary T cells after IL2RG knockin. The right panel shows knockin efficiency across three independent donors (Donors B, and C).

FIG. 11 shows that genomic location affects the expression of the integrated transgene, according to certain aspects of this disclosure. IDLV containing an HDR template that places GFP-P2A upstream of the N-terminal methionine of either ACTB or IL2RG was transduced into human primary T cells and Cas9-sgACTB or Cas9-sgIL2RG RNP was electroporated in 24 hours later. Primary human T cells were assayed 7 days later via flow cytometry. The GFP median fluorescent intensity tracks with the degree of expression of the endogenous locus. ACTB is expressed much higher in primary human T cells than IL2RG, leading to increased expression of the GFP transgene integrated at the ACTB locus.

FIG. 12 shows a comparison of the methods of certain aspects of this disclosure to other methods that could feasibly have equivalent genetic payload size. This includes delivery of the same template that was generated via PCR or delivery of a whole plasmid containing the same HDR template and cutsites. The IDLV method according to certain aspects of this disclosure results in dramatically increased viability relative to the other two methods, which were highly toxic to primary T cells.

FIG. 13 shows that the methods of certain aspects of this disclosure are robust to experimental perturbations in human primary T cells. The top panel shows the results of changing the number of cells in the electroporation reaction from the normal 1 million total cells to 500,000 or 250,000, which did not dramatically change knock-in efficiency. The bottom panel shows the results of changing the time of transduction from 24 hours before Cas9 RNP electroporation to 48, which did not dramatically change knock-in efficiency.

FIG. 14 shows a method for ensuring stable expression of large, hard to express, and/or easily silenced transgenes, according to certain aspects of this disclosure. Transgenes introduced using traditional viral methods of genetic engineering methods are prone to silencing. Knocking in a transgene upstream of an essential gene (such as ACTB) along with a polycistronic element (e.g., a P2A element or IRES) stabilizes gene expression by creating a selection pressure against transgene silencing.

FIG. 15 shows design (top panel) and analysis (bottom panel) of a specific knock-in system with HA that enables integration of mCherry and RfxCas13d directly upstream of the N-terminal methionine on beta-actin (ACTB gene), according to certain aspects of this disclosure. The design is packaged into an IDLV. The nuclease system is CRISPR-Cas9 that uses a single guide RNA to cut both the HDR template twice and genomic ACTB (sgACTB). P2A is used to separate mCherry, RfxCas13d, and ACTB from each other post-translation. Primary human T cells were transduced with the IDLV, electroporated with Cas9-sgACTB RNP 24 hours later, and assayed 7 days post-electroporation via flow cytometry. In parallel, cells were transduced with lentivirus driving Cas13d expression with either the EF1α or SFFV promoters. Integration of Cas13d into the essential gene, ACTB, stabilizes gene expression over time whereas Cas13d integrated using traditional, randomly integrating lentivirus was silenced dramatically over time.

FIG. 16 shows use of the method according to certain aspects of this disclosure to integrate RfCas13d into the ACTB locus of K562 cells. CRISPR RNA driven by a U6 promoter was then lentivirally introduced into the cells. The integrated transgenes are fully functional, as cells receiving a CRISPR RNA targeted to the CD46 transcript (crCD46) expressed less surface CD46 (as measured by flow cytometry) than cells without CRISPR RNA or cells containing a non-targeting CRISPR RNA (crNT).

FIG. 17 shows the design (top panel) and analysis (bottom panel) of a specific knock-in system with HA that enables integration of dCas12a-VPR (˜5.7 kb) and GFP directly upstream of the N-terminal methionine on beta-actin (ACTB gene), demonstrating successful knock-in of large transgenes, according to certain aspects of this disclosure. The design is packaged into an IDLV. The nuclease system is CRISPR-Cas9 that uses a single guide RNA to cut both the HDR template twice and genomic ACTB (sgACTB). P2A is used to separate GFP, dCas12a-VPR, and ACTB from each other post-translation. K562 cells were transduced with the IDLV, electroporated with Cas9-sgACTB RNP 24 hours later, and assayed 7 days post-electroporation via flow cytometry.

FIG. 18 shows a comparison of the methods according to certain aspects of this disclosure to traditional lentiviral methods. The top panel shows the results of primary human T cells transduced with lentivirus driving dCas12a-VPR expression with either the EF1α or SFFV promoters and assayed after 3 days. In this period of time, the cells had already completely silenced the gene. The bottom panel shows the results of using an embodiment of the methods described herein to integrate dCas12a-VPR into ACTB, enabling long-term stable expression of the transgene, even when traditional lentiviral method had already silenced this difficult to express gene.

FIG. 19 shows the design (top panel) and analysis (bottom panel) of a specific knock-in system with HA that enables integration of the SARS-CoV-2 Spike protein S1 subunit, a highly conserved fragment of the SARS-CoV-2 RNA dependent RNA polymerase (RdRP), and GFP directly upstream of the N-terminal methionine on beta-actin (ACTB gene), according to certain aspects of this disclosure. The design is packaged into an IDLV. The nuclease system is CRISPR-Cas9 that uses a single guide RNA to cut both the HDR template twice and genomic ACTB (sgACTB). P2A, E2A, and T2A is used to separate GFP, dCas12a-VPR, and ACTB from each other post-translation. Primary human T cells were transduced with the IDLV, electroporated with Cas9-sgACTB RNP 24 hours later, and analyzed 3, 9, and 15 days post-electroporation via flow cytometry. In parallel, cells were transduced with lentivirus driving SARS-CoV-2 protein expression with either the EF1α or SFFV promoters.

FIG. 20 shows that the method according to certain aspects of this disclosure creates higher expression of an integrated transgene than more traditional lentiviral methods. Primly human T cells were transduced with IDLV (as shown in FIG. 19, top panel), electroporated with Cas9-sgACTB RNP 24 hours later, and assayed 3 days post-electroporation via flow cytometry.

FIG. 21 shows that integration of payload transgenes at essential endogenous gene loci stabilizes transgene expression, according to certain aspects of this disclosure. The toxic S1 domain from SARS-CoV-2 Spike protein, SARS-CoV-2 RNA dependent RNA polymerase, and GFP (5.7 kb) was knocked in upstream of ACTB under the control of the endogenous promoter using a method described herein or under the control of a synthetic promoter (EF1α) using traditional lentiviral methods. Transgenes integrated according to traditional methods were silenced over a two-week period, while transgenes integrated according to the method described in certain aspects of this disclosure remained stable.

FIG. 22 shows the results of Jurkat cells transduced with the IDLV (as shown in FIG. 19, top panel) and electroporated with Cas9-sgACTB RNP, according to certain aspects of this disclosure. The transduced cells were then submitted for immunopeptidotnics to see if the peptide was being presented on MHCI. The assay revealed two peptides in RdRP that were presented by MHCI and were also predicted to be strong hinders of the Jurkat's HLA type.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Introduction

The present disclosure is based, in part, on two discoveries: 1) that addition of cleavage sites to homologous recombination repair templates (donor templates) enables more efficient transgene knock-in (integration of transgene into target genome), and 2) that integration of a transgene at an endogenous gene locus (e.g., a gene encoding a product that is essential for cell survival) promotes stable, high, long-term transgene expression.

The methods and compositions disclosed herein provide a number of advantages, including but not limited to the following: efficient knock-in of large transgene payloads (e.g., greater than 4,000 nucleotides); increased viability in transduced cells relative to traditional methods; integration of payloads into precise genomic loci; integration of multiple payloads into multiple genomic loci at once; long-term stable expression of integrated transgenes; and high expression of integrated transgenes.

### II. Compositions and Methods of Use of Certain Embodiments

Disclosed herein are some embodiments of compositions, systems, and methods for use in genome editing. In some instances, the methods comprise delivery of a payload to a host cell and integration of the payload into the genome of the host cell at a desired locus. As used herein, the term “payload” refers to a nucleotide sequence which is inserted into the genome of a host cell. In some embodiments, the payload may be any length up to 12,000 nucleotides (nt). For example, the payload may be up to 500 nt, up to 1,000 nt, up to 2,000 nt, up to 4,000 nt, up to 4,400 nt, up to 5,000 nt, up to 7,000 nt, up to 8,000 nt, up to 8.500 nt, up to 10,000 nt, up to 11,000 nt, or up to 12,000 nt. In one embodiment, the payload may be up to 4,400 nt. In another embodiment, the payload may be up to 4,700 nt. In another embodiment, the payload may be up to 8,000 nt. In another embodiment, the payload may be up to 8,500 nt.

In some embodiments, the payload may be at least 100 nt. For example, the payload may be at least 500 nt, at least 1,000 nt, at least 2,000 nt, at least 4,000 nt, at least 4,400 nt, at least 5,000 nt, at least 6,000 nt, at least 7,000 nt, at least 8,000 nt, at least 8,500 nt, at least 9,000 nt, at least 10,000 nt, at least 11,000 nt, or at least 11,500 nt. In one embodiment, the payload comprises a nucleotide sequence of at least 4,400 nt. In another embodiment, the payload comprises a nucleotide sequence of at least 4,700 nt. In another embodiment, the payload comprises a nucleotide sequence of at least 6,000 nt.

In some embodiments, the payload comprises a gene or transgene which can be expressed in the host cell. In some instances, the compositions, systems, and methods disclosed herein comprise nuclease systems targeting the desired locus, donor templates or vectors for inserting the payload, and viruses or pseudoviruses comprising the donor templates or vectors. Also disclosed herein are methods of using such systems, templates or vectors to produce modified cells that have the payload integrated into the genome at the desired locus. Also disclosed herein are modified cells produced using the described methods and/or compositions, vaccines comprising the modified cells, and methods of using the modified cells or vaccines to induce an immune response in a subject.

In some instances, delivery of the payload to the desired locus can be accomplished through methods such as homologous recombination. As used herein, “homologous recombination (HR)” refers to insertion of a nucleotide sequence during repair of double-strand breaks in DNA via homology-directed repair mechanisms. This process uses a “donor” molecule or “donor template” with homology to nucleotide sequence in the region of the break as a template for repairing a double-strand break. The presence of a double-stranded break facilitates integration of the donor sequence. The donor sequence may be physically integrated or used as a template for repair of the, break via homologous recombination, resulting in the introduction of all or part of the nucleotide sequence. This process is used by a number of different gene editing platforms that create the double-strand break, such as meganucleases, zinc finger nucleases (ZFNs), transcription activator-like effector nucleases (TALENs), Argonautes, and the CRISPR-Cas gene editing systems. In some instances, the payload can be inserted at the desired locus through mechanisms which do not involve a nuclease (e.g., a protein which can bind to the desired locus and produce R-loops or D-loops).

In some embodiments, payloads are delivered to two or more loci. For example, two payloads comprising the same or different transgenes may be integrated, or one of the payloads may comprise a first gene and the second payload may comprise a second gene that acts as a synthetic regulator of the first gene or that acts to bias the modified cells towards a certain lineage (e.g., by expressing a transcription factor from the second locus). In some embodiments, one payload is delivered to two or more loci. In some embodiments, at least two different payloads are delivered to at least two loci.

In some embodiments, payloads comprising a transgene without a promoter are integrated into an endogenous gene such that expression of the transgene is driven by the endogenous promoter. In some embodiments, these transgene payloads comprise a polycistronic expression element allowing translation of both the endogenous gene and the transgene from a single mRNA transcript produced from the endogenous promoter. In some embodiments, payloads comprising a transgene without a promoter are targeted for insertion into a gene which produces a product essential for cell viability. In such instances, silencing of the transgene may lead to cell death.

Also provided herein are modified cells produced using the methods or compositions described. As used herein, a “cell”, “modified cell” or “modified host cell” refers to a population of cells descended from the same cell or from the same initial population of cells, with each cell of the population having a similar genetic make-up and retaining the same modification. Also provided herein are methods of using the modified cells and/or vaccines comprising such modified cells to produce an immune response in a subject.

In some embodiments, the methods provided herein result in transduced cells having improved viability relative to cells transduced using traditional methods (e.g., transduction with traditional lentiviral vectors, transfection with recombination templates in plasmid backbones or as PCR products, etc.), as demonstrated, e.g., in the Examples herein. In some embodiments, the methods herein result in transduced cells with improved or prolonged transgene expression (i.e., stabilized transgene expression) relative to cells transduced using traditional methods, as demonstrated, e.g., in the Examples herein. In some embodiments, stabilized transgene expression is achieved for large and/or difficult to express (e.g., due to cellular toxicity) transgenes (e.g., stabilized expression of Cas13d and dCas12a-VPR, as demonstrated in Example 9 herein).

### III. Compositions and Methods for Making Modified Cells

### A. Cells

Disclosed herein, in some embodiments, are compositions comprising modified host cells, preferably human cells, that have a payload inserted into at least one genomic locus. In some embodiments, the payload comprises a transgene. Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is a lymphocyte, a phagocytic cell (e.g., a CD14+ monocyte, a CD16+ H monocyte, or a macrophage), a granulocytic cell (e.g., a neutrophil, a basophil, an eosinophil, or a mast cell), or a dendritic cell (e.g., a cDC1, a cDC2, a pDC, a tDC, or a monocyte derived DC). In some embodiments, the cell is an embryonic stem cell, a stem cell, a pluripotent stem cell, an induced pluripotent stem (iPS) cell, a somatic stem cell, an adult stem cell, a differentiated cell, a mesenchymal stem cell or a mesenchymal stromal cell, a neural stem cell, a hematopoietic stem cell, an adipose stem cell, a keratinocyte, a skeletal stem cell, or a muscle stem cell. In some embodiments, the cell is a progenitor cell, a hematopoietic progenitor cell, a neural progenitor cell, a skeletal progenitor cell, a muscle progenitor cell, a fat progenitor cell, a heart progenitor cell, a chondrocyte, or a pancreatic progenitor cell. In some embodiments, the cell is a fibroblast, a natural killer (NK) cell, a B-cell (including plasma cells), an invariant natural killer (iNKT) cell, a T cell (e.g., a CD4+ helper T cell, a CDS830 T cell, a δγ T cell, or a Natural Killer (NKT) cell), an innate lymphoid cell (ILC) (e.g., a Group 1 ILC, a Group 2 ILC, or a Group 3 ILC), or a peripheral blood mononuclear cell (PBMC). For example, the cell may be engineered to express a chimeric antigen receptor (CAR), thereby creating a CAR-T cell. In some embodiments, the cell lines are T cells that have at least one payload inserted into at least one genomic locus. In some embodiments, the payload comprises a transgene which expresses a CAR. In some embodiments, CAR-T cells produced using the methods and compositions provided herein can be used in therapy (e.g., cancer immunotherapy). In some embodiments, the modified cell produced using the methods and compositions disclosed herein may express a viral antigen (e.g., SARS-CoV-2 Spike protein or SARS-CoV-2 RNA dependent RNA polymerase protein). In some embodiments, e.g., as demonstrated in Example 9 herein, the viral antigen may be expressed on the surface of the modified cell or presented by the cell on major histocompatibility complex I or II (MHCI or MHCII). In some embodiments, a modified cell expressing a viral antigen on the surface may be administered to a patient to induce an immune response. In some embodiments, the cell lines are pluripotent stem cells that have at least one payload inserted into at least one genomic locus.

To prevent immune rejection of the modified cells when administered to a subject, the cells to be modified are preferably derived from the subject's own cells. Thus, preferably the mammalian cells are from the subject to be treated with the modified cells. In some instances, the mammalian cells are modified to be autologous cells. In some instances, the mammalian cells are further modified to be allogeneic cells. In some instances, modified T cells can be fiirther modified to be allogeneic, for example, by inactivating the T cell receptor locus. In some instances, modified cells can further be modified to be allogeneic, for example, by deleting B2M to remove MHC class I on the surface of the cell, or by deleting B2M and then adding back an HLA-G-B2M fusion to the surface to prevent NK cell rejection of cells that do not have MHC Class I on their surface.

For example, the cells may be stem cells isolated from the subject for use in a regenerative medical treatment in any of epithelium, cartilage, hone, smooth muscle, striated muscle, neural epithelium, stratified squamous epithelium, and ganglia. Disease that results from the death or dysfunction of one or a few cell types, such as Parkinson's disease and juvenile onset diabetes, are also commonly treated using stem cells (see, Thomson et al., Science, 282:1145-1147, 1998, which is hereby incorporated by reference in its entirety).

In some embodiments, cells are harvested from the subject and modified according to the methods disclosed herein, which can include selecting certain cell types, optionally expanding the cells and optionally culturing the cells, and which can additionally include selecting cells that contain the at least one payload inserted into the at least one genomic locus.

Also disclosed herein are vaccines and therapeutic compositions comprising a modified cell of the present disclosure. The vaccines and therapeutic compositions may comprise a pharmaceutically acceptable carrier (excipient). A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The pharmaceutical compositions may further comprise a diluent, solubilizer, emulsifier, preservative, and/or adjuvant to be used with the methods disclosed herein. Suitable carriers and their formulations are described in Remington: The Science and Practice of Pharmacy, 21st Edition, Philip P. Gerbino, ed., Lippincott Williams & Wilkins (2006).

### B. Donor Templates or Vectors for Inserting the Payload

In some embodiments, the compositions disclosed herein comprise donor templates or vectors for inserting at least one payload into at least one genomic locus.

In some embodiments, the donor template comprises (a) one or more nucleotide sequences homologous to a fragment of the desired locus, or homologous to the complement of said locus, (b) a payload optionally comprising a transgene, optionally linked to an expression control sequence, and (c) one or more cleavage sites comprising nucleotide sequences that can be bound or cleaved by a nuclease. In some embodiments, the cleavage sites are homologous to a fragment of the desired locus, or homologous to the complement of said locus. In such instances, a nuclease system may be able to cleave DNA at both the endogenous locus and in the donor template. In some embodiments, after a nuclease system is used to cleave DNA, introduction of a donor template can take advantage of homology-directed repair mechanisms to insert the payload sequence during repair of the break in the DNA. In some instances, the donor template comprises a region that is homologous to nucleotide sequence in the region of the break (referred to herein as a “homology arm”) so that the donor template hybridizes to the region adjacent to the break and is used as a template for repairing the break. In instances where the donor template comprises cleavage sites which are bound or cleaved by a nuclease, the payload sequence may be more effectively inserted at the desired locus.

In some embodiments, the payload is flanked on both sides by homology arms that are homologous to a fragment of the desired locus or the complement thereof. In some embodiments, the payload is flanked on both sides by cleavage sites which may be homologous to a fragment of the desired locus or the complement thereof. In a preferred embodiment, the donor template comprises at least two cleavage sites, at least two homology arms, and a payload arranged according to the following linear order: cleavage site 1, homology arm 1, payload, homology arm 2, cleavage site 2. In some embodiments, cleavage sites 1 and 2 comprise the same nucleotide sequence.

In some embodiments, the donor template comprises more than one payload. Such a donor template may be used to insert multiple payloads at multiple genomic sites. For example, the donor template may comprise two payloads, which may comprise two different nucleotide sequences or the same nucleotide sequence, flanked by two different sets of homology arms that are homologous to fragments of each desired insertion locus or the complements thereof. In some embodiments, the payloads are flanked by cleavage sites that are homologous to fragments of each desired insertion locus or the complements thereof. In a preferred embodiment, the donor template comprises two payloads, four homology arms, and four cleavage sites arranged according to the following linear order: cleavage site 1, homology arm 1, payload 1, homology arm 2, cleavage site 2, cleavage site 3, homology arm 3, payload 2, homology arm 4, cleavage site 4. In some embodiments, cleavage sites I and 2 comprise the same nucleotide sequence, and cleavage sites 3 and 4 comprise the same nucleotide sequence,

In some embodiments, the payload comprises a transgene. As used herein, the term “transgene” refers to a gene which is artificially introduced into the genome of an organism. In some embodiments, the transgene comprises a coding sequence. As used herein, a “coding sequence” or a sequence which “encodes” a product is a nucleic acid molecule which is transcribed (in the case of DNA) and translated (in the case of messenger RNA) into a product in vivo when placed under the control of appropriate control elements. For example, a DNA coding sequence may be transcribed into an RNA product, which may be functional as an RNA molecule (e.g., a long noncoding RNA or transfer RNA). Alternatively, the RNA product may itself be a coding sequence (e.g., messenger RNA) for a polypeptide product. The boundaries of the coding sequence can be determined by a start codon at the 5′ (amino) terminus and a translation stop codon at the 3′ (carboxy) terminus. A coding sequence can include, but is not limited to, complementary DNA (cDNA) from viral, prokaryotic, or eukaryotic messenger RNA, genomic DNA sequences from viral or prokaryotic DNA, and even synthetic DNA sequences. A transcription termination sequence may be located 3′ to the coding sequence.

Typical “control elements” include, but are not limited to, transcription promoters (which may include inducible promoters, constitutive promoters, and tissue-specific promoters), transcription enhancer elements, transcription termination signals, polyadenylation sequences (located 3′ to the translation stop codon), sequences for optimization of initiation of translation (located 5′ to the coding sequence), translation enhancement sequences, and translation termination sequences. In some embodiments, any control elements present in the payload are operably linked to a coding sequence. As used herein, “operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

In some instances, the payload described herein comprises a promoter operably linked to a coding sequence. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982) 79:6777 and elements derived from human CMV, as described in Boshart et al., Cell (1985) 41:521, such as elements included in the CMV intron A sequence.

In some embodiments, the payload described herein does not comprise a promoter. Such payloads may be integrated into a genomic locus in a host cell such that an endogenous promoter is operably linked to the coding sequence of the payload (i.e., a promoter endogenous to the host cell drives transcription of the coding sequence). In some embodiments, a payload that does not comprise a promoter may comprise one or more polycistronic elements. As used herein, “polycistronic element” refers to a sequence element which allows translation of multiple polypeptide products from a single mRNA transcript. The polycistronic elements may include an internal ribosome entry site (IRES) or a 2A self-cleaving peptide element (e.g., T2A, P2A, E2A, or F2A). In some embodiments, the polycistronic element allows an endogenous promoter to drive expression of both the transgene and the endogenous gene at which the transgene is integrated. In some embodiments, the payload transgene lacking a promoter is integrated at an endogenous gene that is essential for cell survival. This may promote long-term, stable expression, because any silencing of the integrated transgene will also lead to silencing of the essential endogenous gene. In some embodiments, then, such a strategy may promote survival of cells which do not silence the integrated transgene.

In some instances, the donor polynucleotide or vector comprising a payload comprising a transgene optionally further comprises an expression control sequence operably linked to said transgene.

In some instances, the donor template is single stranded, double stranded, a plasmid, a DNA fragment, or a vector.

In some instances, donor template plasmids comprise additional elements necessary for replication, including a promoter and optionally a 3′ UTR.

In some instances, donor template vectors comprise additional elements necessary for replication, transcription, or reverse transcription of the vector.

The vector can be a viral vector, such as a retroviral, pseudoviral, lentiviral (both integration competent and integration defective lentiviral vectors), adenoviral, adeno-associated viral or herpes simplex viral vector. The viral vector may also be an Alphaviral, flaviviral, Rhabdoviral, Newcastle disease viral, Picornaviral, poxviral, Coxsackieviral, or measles viral vector. Viral vectors may further comprise genes necessary for replication, transcription, or reverse transcription of the viral vector. In some embodiments, the vector is a modified viral vector a single coding gene or regulatory element sequence on the viral vector has been changed relative to its reference sequence).

In some embodiments, the donor template comprises: (1) a viral vector backbone, e.g. a lentiviral backbone, to generate virus; (2) cleavage sites that can be bound or cleaved by a nuclease; (3) arms of homology to the target site of 100 base pairs (bp) to 1000 bp (e.g., around 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, or 950 bp) on each side to assure high levels of reproducible targeting to the site (see, Porteus, Annual Review of Pharmacology and Toxicology, Vol. 56:163-190 (2016); which is hereby incorporated by reference in its entirety); (4) a payload optionally comprising a transgene with an optional expression control sequence operably linked to the transgene; and (5) an optional additional marker gene to allow for enrichment and/or monitoring of the modified host cells.

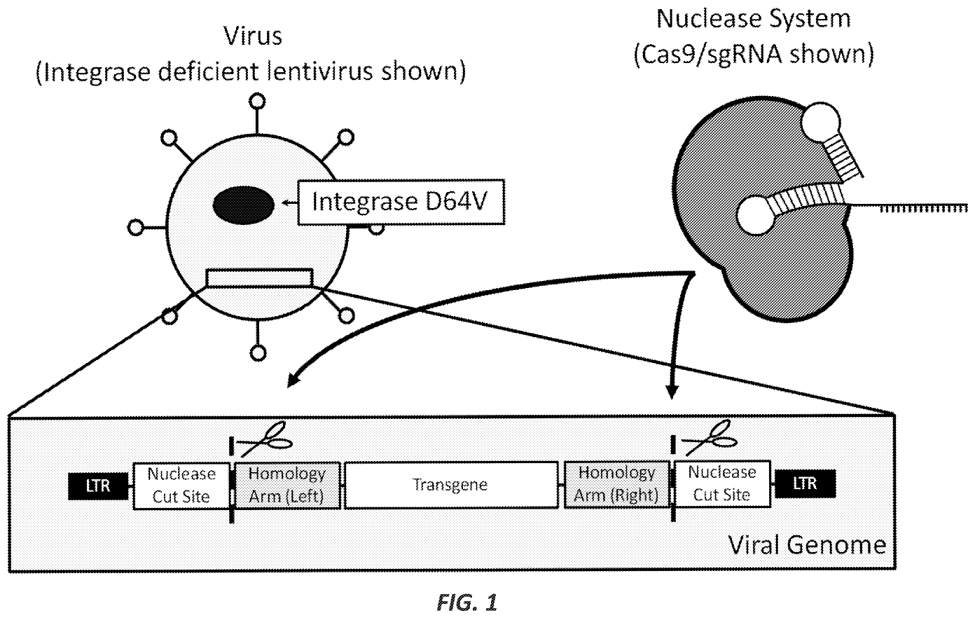

In a particular embodiment, as shown in FIG. 1, the donor template comprises a viral vector backbone, e.g. a lentiviral backbone with an integrase gene encoding a mutant integrase with a D64V substitution, to generate integrase deficient lentivirus; (2) cleavage sites that can be bound or cleaved by a nuclease (e.g., Cas9); (3) homology aims; and (4) a payload comprising a transgene.

Suitable marker genes are known in the art and include Myc, HA, FLAG, GFP, mCherry, truncated NGFR, truncated EGFR, truncated CD20, truncated CD19, as well as antibiotic resistance genes.

Any lentivirus known in the art can be used. In some embodiments, the lentivirus is integration-deficient. In some embodiments, the integration-deficient lentivirus comprises a mutant integrase protein comprising a D64V substitution. In some embodiments, the integration-deficient lentivirus is produced using the plasmid sequence of SEQ NO: 1.

In any of the preceding embodiments, the donor template or vector may comprise a nucleotide sequence substantially identical to a fragment of the desired locus, wherein the nucleotide sequence is at least 85%, 88%, 90%, 92%, 95%, 98%, or 99% identical to 100-1000 consecutive nucleotides (e.g., at least 100, 200, 250, 300, 350, 400, 450, 500, 550, 600, 650, 700, 750, 800, 850, 900, or 950 consecutive nucleotides) of the desired locus; around 400 nucleotides is usually sufficient to assure accurate recombination. In some embodiments, the desired locus comprises a gene essential for cell survival, including but not limited to beta-actin, cytochrome P450 (POR), or ribosomal subunit S19 (RPS19). In some embodiments, the desired locus comprises a gene essential for survival of a particular cell type, including but not limited to IL2 receptor gamma (IL2RG) or CD3 epsilon chain (CD3e). In some embodiments, the desired locus comprises a gene with a high expression level and/or a positive relationship with cell growth. In some embodiments, the desired locus comprises a cell-type specific gene, including but not limited to an oncogene, a tumor suppressor gene, or a lineage marker gene.

The disclosure herein also provides viruses or pseudoviruses comprising the donor template or vector described above. In some embodiments, the virus or pseudovirus (e.g., lentivirus) is integration deficient. In some embodiments, the pseudovirus is a lentivirus comprising the donor template or vector described above between long terminal repeats (LTRs) in the lentiviral genome. In some embodiments, the described viruses or pseudoviruses are useful for delivering the donor template or vector to host cells as described herein.

The disclosure herein also contemplates methods and systems for targeting integration of a payload to a desired locus comprising said donor template or vector and a nuclease targeted to said locus. In some embodiments, the nuclease is a CRISPR-associated (Cas) protein. In some embodiments, the system further comprises a guide nucleic acid which serves to target the Cas protein to the desired locus.

The disclosure herein further contemplates methods and systems for targeting integration of a payload to a desired locus comprising said donor template or vector and a nuclease specific for said locus. The nuclease can be, for example, a meganuclease, a ZFN, a TALEN, an Argonaute protein, or a transposase protein.

### C. Nuclease

Any suitable nuclease can be used in the systems and methods disclosed herein. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof any derivative thereof; any variant thereof; and any fragment thereof.

A nuclease as disclosed herein can be coupled (e.g., linked or fused) to additional peptide sequences which are not involved in regulating gene expression, for example linker sequences, targeting sequences, etc. The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a nuclease) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

In a preferred embodiment, a nuclease as disclosed herein comprises an NLS. Non-limiting examples of NLSs include an NLS sequence derived from: the NLS of the SV40 virus large T-antigen, having the amino acid sequence PKKKRKV (SEQ ID NO: 2); the NLS from nucleoplasmin (e.g. the nucleoplasmin bipartite NLS with the sequence KRPAATKKAGQAKKKK (SEQ ID NO: 3)); the c-myc NLS having the amino acid sequence PAAKRVKLD (SEQ ID NO: 4) or RQRRNELKRSP (SEQ ID NO: 5); the hRNPA1 M9 NLS having the sequence NQSSNFGPMKGGNFGGRSSGPYGGGGQYFAKPRNQGGY (SEQ ID NO: 6); the sequence RMRIZFKNKGKDTAELRRRRVEVSVELRKAKKDEQILKRRNV (SEQ ID NO: 7) of the IBB domain from importin-alpha; the sequences VSRKRPRP (SEQ ID NO: 8) and PPKKARED (SEQ ID NO: 9) of the myoma T protein; the sequence PQPKKKPL (SEQ ID NO: 10) of human p53; the sequence SALIKKKKKMAP (SEQ ID NO: 11) of mouse c-abl IV; the sequences DRLRR (SEQ ID NO: 12) and PKQKKRK (SEQ ID NO: 13) of the influenza virus NS1; the sequence RKLKKKIKKL (SEQ ID NO: 14) of the Hepatitis virus delta antigen; the sequence REKKKFLKRR, (SEQ ID NO: 15) of the mouse Mx1 protein; the sequence KRKGDEVDGVDEVAKKKSKK (SEQ ID NO: 16) of the human poly(ADP-ribose) polymerase, and the sequence RKCLQAGMNLEARKTKK (SEQ ID NO: 17) of the steroid hormone receptors (human) glucocorticoid.

In some embodiments, the nuclease can be complexed with at least one guide nucleic acid polynucleotide as described herein. In some embodiments, the at least one guide nucleic acid polynucleotide can be either heterologous DNA polynucleotide or heterologous RNA polynucleotide. In some cases, the complexing with the at least one heterologous RNA polynucleotide directs and targets the nuclease to the portion of the genome (e.g., mammalian genome or human genome) targeted for insertion of the payload.

In some embodiments, the nuclease comprises a CRISPR-associated (Cas) protein or a Cas nuclease which functions in a non-naturally occurring CRISPR (Clustered Regularly Interspaced Short Palindromic Repeats)/Cas (CRISPR-associated) system. In bacteria, this system can provide adaptive immunity against foreign DNA (Barrangou, R., et al, “CRISPR provides acquired resistance against viruses in prokaryotes, “Science (2007) 315: 1709-1712; Makarova, K. S., et al, “Evolution and classification of the CRISPR-Cas systems,” Nat Rev Microbiol (2011) 9:467-477; Garneau, J. E., et al, The CRISPR/Cas bacterial immune system cleaves bacteriophage and plasmid DNA,” Nature (2010) 468:67-71; Sapranauskas, R., et al, “The Streptococcus thermophilus CRISPR/Cas system provides immunity in Escherichia coli,” Nucleic Acids Res (2011) 39: 9275-9282).

In a wide variety of organisms including diverse mammals, animals, plants, microbes, and yeast, a CRISPR/Cas system (e.g., modified and/or unmodified) can be utilized as a genome engineering tool. A CRISPR/Cas system can comprise a guide nucleic acid such as a guide RNA (gRNA) complexed with a Cas protein for targeted regulation of gene expression and/or activity or nucleic acid editing. An RNA-guided Cas protein (e.g., a Cas nuclease such as a Cas9 nuclease) can specifically bind a target polynucleotide (e.g., DNA) in a sequence-dependent manner. The Cas protein, if possessing nuclease activity, can cleave the DNA (Gasiunas, G., et al, “Cas9-crRNA ribonucleoprotein complex mediates specific DNA cleavage for adaptive immunity in bacteria,” Proc Natl Acad Sci USA (2012) 109: E2579-E2 86; Jinek, M., et al, “A programmable dual-RNA-guided DNA endonuclease in adaptive bacterial immunity,” Science (2012) 337:816-821; Sternberg, S. H., et al, “DNA interrogation by the CRISPR RNA-guided endonuclease Cas9,” Nature (2014) 507:62, Deltcheva, E., et al, “CRISPR RNA maturation by trans-encoded small RNA and host factor RNase III,” Nature (201 1) 471:602-607), and has been widely used for programmable genome editing in a variety of organisms and model systems (Cong, L., et al, “Multiplex genome engineering using CRISPR Cas systems,” Science (2013) 339:819-823; Jiang, W., et al, “RNA-guided editing of bacterial genomes using CRISPR-Cas systems,” Nat. Biotechnol. (2013) 31: 233-239; Sander, J. D. & Joung, J. K, “CRISPR-Cas systems for editing, regulating and targeting genomes,” Nature Biotechnol. (2014) 32:347-355).

In some cases, the Cas protein is mutated and/or modified to yield a nuclease deficient protein or a protein with decreased nuclease activity relative to a wild-type Cas protein. A nuclease deficient protein can retain the ability to bind DNA, but may lack or have reduced nucleic acid cleavage activity. A Cas nuclease (e.g., retaining wild-type nuclease activity, having reduced nuclease activity, and/or lacking nuclease activity) can function in a CRISPR/Cas system to regulate the level and/or activity of a target gene or protein (e.g., decrease, increase, or elimination). The Cas protein can bind to a target polynucleotide and prevent transcription by physical obstruction or edit a nucleic acid sequence to yield non-functional gene products. A Cas protein can edit a nucleic acid sequence by generating a double-stranded break or single-stranded break in a target polynucleotide. A double-strand break in DNA can result in DNA break repair which allows for the introduction of gene modification(s) (e.g., nucleic acid editing). DNA break repair can occur via non-homologous end joining (NHEJ) or homology-directed repair (HDR). In HDR, a donor DNA repair template or template polynucleotide that contains homology arms flanking sites of the target DNA, as described herein, can be provided.

In some embodiments, the nuclease described herein comprises a Cas protein that forms a complex with a guide nucleic acid, such as a guide RNA. In some embodiments, the nuclease comprises a Cas protein that forms a complex with a single guide nucleic acid, such as a single guide RNA (sgRNA). In some embodiments, the nuclease comprises a RNA-binding protein (RBP) optionally complexed with a guide nucleic acid, such as a guide RNA (e.g., sgRNA), which is able to form a complex with a Cas protein. In some embodiments, the nuclease comprises a nuclease-null DNA binding protein derived from a DNA nuclease that can induce transcriptional activation or repression of a target DNA sequence. In some embodiments, the nuclease comprises a nuclease-null RNA binding protein derived from a RNA.

Any suitable CRISPR/Cas system can be used. A CRISPR/Cas system can be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. A CRISPR/Cas system can be a type I, a type II, a type III, a type IV, a type V, a type VI system, or any other suitable CRISPR/Cas system. A CRISPR/Cas system as used herein can be a Class 1, Class 2, or any other suitably classified CRISPR/Cas system. Class 1 or Class 2 determination can be based upon the genes encoding the effector module. Class 1 systems generally have a multi-subunit crRNA-effector complex, whereas Class 2 systems generally have a single protein, such as Cas9, Cpf1, C2c1, C2c2, C2c3 or a crRNA-effector complex. A Class 1 CRISPR/Cas system can use a complex of multiple Cas proteins to effect regulation. A Class 1 CRISPR/Cas system can comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. A Class 2 CRISPR/Cas system can use a single large Cas protein to effect regulation. A Class 2 CRISPR/Cas systems can comprise, for example, type II (e.g., II, IIA, IIB) and type V CRISPR/Cas type. CRISPR systems can be complementary to each other, and/or can lend functional units in trans to facilitate CRISPR locus targeting.

A nuclease comprising a Cas protein can be a Class 1 or a Class 2 Cas protein. A Cas protein can be a type I, type II, type III, type IV, type V Cas protein, or type VI Cas protein. A Cas protein can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A Cas protein can be a chimeric Cas protein that is fused to other proteins or polypeptides. A Cas protein can be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas1Od, Cas10, Cas1Od, CasG, CasH, Cpf1, Csy1, Csy2, Csy3, Cse1 (CasA), Cse2 (CasB), Cse3 (CasE), Cse4 (CasC), Csc1, Cse2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

A Cas protein can be from any suitable organism. Non-limiting examples include Streptococcus pyogenes, Streptococcus thermophilus, Streptococcus sp. Staphylococcus aureus, Nocardiopsis dassonvillei, Streptomyces pristinae spiralis, Streptomyces viridochromogenes, Streptomyces viridochromogenes, Streptosporangium roseum, Streptosporatigium roseum, AlicyclobacHlus acidocaldarius, Bacillus pseudomycoides, Bacillus selenitireaucens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

A Cas protein can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractorsalsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseohacter shibae, Azospirillum, Nitrobacter hamburgensis, Bradyrhizobium, Wolinellasuccinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

A Cas protein as used herein can be a wild-type or a modified form of a Cas protein. A Cas protein can be an active variant, inactive variant, or fragment of a wild-type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild-type exemplary Cas protein. A Cas protein can be a polypeptide with at most about 5%, 10%, 20%, 30%, 40%, 50%, 60%, 70%, 80%, 90%, 100% sequence identity and/or sequence similarity to a wild-type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild-type or modified Cas protein or a portion thereof. Variants or fragments can be targeted to a nucleic acid locus in complex with a guide nucleic acid while lacking nucleic acid cleavage activity.

A Cas protein can comprise one or more nuclease domains, such as DNase domains. For example, a Cas9 protein can comprise a RuvC-like nuclease domain and/or an HNH-like nuclease domain. The RuvC and HNH domains can each cut a different strand of double-stranded DNA to make a double-stranded break in the DNA. A Cas protein can comprise only one nuclease domain (e.g., Cpf1 comprises RuvC domain but lacks HNH domain).

A Cas protein can comprise an amino acid sequence having at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a nuclease domain (e.g., RuvC domain, HNH domain) of a wild-type Cas protein.

A Cas protein can be modified to optimize regulation of gene expression. A Cas protein can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein for regulating gene expression.

A Cas protein can be a fusion protein. For example, a Cas protein can be fused to a cleavage domain, an epigenetic modification domain, a transcriptional activation domain, or a transcriptional repressor domain. A Cas protein can also be fused to a heterologous polypeptide providing increased or decreased stability. The fused domain or heterologous polypeptide can be located at the N-terminus, the C-terminus, or internally within the Cas protein.

A Cas protein can be provided in any form. For example, a Cas protein can be provided in the form of a protein, such as a Cas protein alone or complexed with a guide nucleic acid. A Cas protein can be provided in the form of a nucleic acid encoding the Cas protein, such as an RNA (e.g., messenger RNA (mRNA)) or DNA. The nucleic acid encoding the Cas protein can be codon optimized for efficient translation into protein in a particular cell or organism.

Nucleic acids encoding Cas proteins can be stably integrated in the genome of the cell. Nucleic acids encoding Cas proteins can be operably linked to a promoter active in the cell. Nucleic acids encoding Cas proteins can be operably linked to a promoter in an expression construct. Expression constructs can include any nucleic acid constructs capable of directing expression of a gene or other nucleic acid sequence of interest (e.g., a Cas gene) and which can transfer such a nucleic acid sequence of interest to a target cell.

In some embodiments, a Cas protein is a dead Cas protein. A dead Cas protein can be a protein that lacks nucleic acid cleavage activity.

A Cas protein can comprise a modified form of a wild-type Cas protein. The modified form of the wild-type Cas protein can comprise an amino acid change (e.g., deletion, insertion, or substitution) that reduces the nucleic acid-cleaving activity of the Cas protein. For example, the modified form of the Cas protein can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

A dCas9 polypeptide can associate with a single guide RNA (sgRNA) to activate or repress transcription of target DNA. sgRNAs can be introduced into cells expressing the engineered chimeric receptor polypeptide. In some cases, such cells contain one or more different sgRNAs that target the same nucleic acid. In other cases, the sgRNAs target different nucleic acids in the cell. The nucleic acids targeted by the guide RNA can be any that are expressed in a cell such as an immune cell. The nucleic acids targeted may be a gene involved in immune cell regulation. In some embodiments, the nucleic acid is associated with cancer. The nucleic acid associated with cancer can be a cell cycle gene, cell response gene, apoptosis gene, or phagocytosis gene. The recombinant guide RNA can be recognized by a CRISPR protein, a nuclease-null CRISPR protein, variants thereof, or derivatives thereof.

Enzymatically inactive can refer to a polypeptide that can bind to a nucleic acid sequence in a polynucleotide in a sequence-specific manner, but may not cleave a target polynucleotide. An enzymatically inactive site-directed polypeptide can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity no more than 1%, no more than 2%, no more than 3%, no more than 4%, no more than 5%, no more than 6%, no more than 7%, no more than 8%, no more than 9%, or no more than 10% activity compared to a wild-type exemplary activity (e.g., nucleic acid cleaving activity, wild-type Cas9 activity).

One or a plurality of the nuclease domains (e.g., RuvC, HNH) of a Cas protein can be deleted or mutated so that they are no longer functional or comprise reduced nuclease activity. For example, in a Cas protein comprising at least two nuclease domains (e.g., Cas9), if one of the nuclease domains is deleted or mutated, the resulting Cas protein, known as a nickase, can generate a single-strand break at a CRISPR RNA (crRNA) recognition sequence within a double-stranded DNA but not a double-strand break. Such a nickase can cleave the complementary strand or the non-complementary strand, but may not cleave both. In some embodiments, double strand break targeting specificity is improved by targeting a nickase to opposite strands at two nearby loci. If a nickase cleaves the single strand at both loci, a double strand break is formed and can be repaired via HR as described herein. If all of the nuclease domains of a Cas protein (e.g., both RuvC and HNH nuclease domains in a Cas9 protein; RuvC nuclease domain in a Cpf1 protein) are deleted or mutated, the resulting Cas protein can have a reduced or no ability to cleave both strands of a double-stranded DNA. An example of a mutation that can convert a Cas9 protein into a nickase is a D10A (aspartate to alanine at position 10 of Cas9) mutation in the RuvC domain of Cas9 from S pyogenes. H939A (histidine to alanine at amino acid position 839) or H840A (histidine to alanine at amino acid position 840) in the HNH domain of Cas9 from S. pyogenes can convert the Cas9 into a nickase. An example of a mutation that can convert a Cas9 protein into a dead Cas9 is a D10A (aspartate to alanine at position 10 of Cas9) mutation in the RuvC domain and H939A (histidine to alanine at amino acid position 839) or H840A (histidine to alanine at amino acid position 840) in the HNH domain of Cas9 from S. pyogenes.

A dead Cas protein can comprise one or more mutations relative to a wild-type version of the protein. The mutation can result in no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity in one or more of the plurality of nucleic acid-cleaving domains of the wild-type Cas protein. The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. The mutation can result in one or inure of the plurality of nucleic acid-cleaving domains lacking the ability to cleave the complementary strand and the non-complementary strand of the target nucleic acid. The residues to be mutated in a nuclease domain can correspond to one or more catalytic residues of the nuclease. For example, residues in the wild-type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated in a nuclease domain of a Cas protein can correspond to residues Asp10, His840, Asn854 and Asn856 in the wild-type S. pyogenes Cas9 polypeptide, for example, as determined by sequence and/or structural alignment.

As non-limiting examples, residues D10, G12, G17, E762, H840, N854, N863, H982, H983, A984, D986, and/or A987 (or the corresponding mutations of any of the Cas proteins) can be mutated. For example, e.g., D10A, G12A, G17A, E762A, H840A, N854A, N863A, H982A, H983A, A984A, and/or D986A. Mutations other than alanine substitutions can be suitable.

A D10A mutation can be combined with one or more of H840A, N854A, or N856A mutations to produce a Cas9 protein substantially lacking DNA cleavage activity (e.g., a dead Cas9 protein). A H840A mutation can be combined with one or more of D10A, N854A, or N856A mutations to produce a site-directed polypeptide substantially lacking DNA cleavage activity. A N854A mutation can be combined with one or more of H840A, D10A, or N856A mutations to produce a site-directed polypeptide substantially lacking DNA cleavage activity. A N856A mutation can be combined with one or more of H840A, N854A, or D10A mutations to produce a site-directed polypeptide substantially lacking DNA cleavage activity.

In some embodiments, a Cas protein is a Class 2 Cas protein. In some embodiments, a Cas protein is a type II Cas protein. In some embodiments, the Cas protein is a Cas9 protein, a modified version of a Cas9 protein, or derived from a Cas9 protein. For example, a Cas9 protein lacking cleavage activity. In some embodiments, the Cas9 protein is a Cas9 protein from S. pyogenes (e.g., SwissProt accession number Q99ZW2). In some embodiments, the Cas9 protein is a Cas9 from S. aureus (e.g., SwissProt accession number J7RUA5). In some embodiments, the Cas9 protein is a modified version of a Cas9 protein from S. pyogenes or S. aureus. In some embodiments, the Cas9 protein is derived from a Cas9 protein from S. pyogenes or S. aureus. For example, a S. pyogenes or S. aureus Cas9 protein lacking cleavage activity.

Cas9 can generally refer to a polypeptide with at least about 5%, 10%, 20%, 30%, 40%, 50%, 60%, 70%, 80%, 90%, 100% sequence identity and/or sequence similarity to a wild-type exemplary Cas9 polypeptide (e.g., Cas9 from S. pyogenes). Cas9 can refer to a polypeptide with at most about 5%, 10%, 20%, 30%, 40%, 50%, 60%, 70%, 80%, 90%, 100% sequence identity and/or sequence similarity to a wild-type exemplary Cas9 polypeptide (e.g., from S. pyogenes). Cas9 can refer to the wildtype or a modified form of the Cas9 protein that can comprise an amino acid change such as a deletion, insertion, substitution, variant, mutation, fusion, chimera, or any combination thereof.

In some embodiments, a nuclease suitable for use in the systems or methods described herein is a “zinc finger nuclease” or “ZFN.” ZFNs refer to a fusion between a cleavage domain, such as a cleavage domain of Fok1, and at least one zinc finger motif (e.g., at least 2, 3, 4, or 5 zinc finger motifs) which can bind polynucleotides such as DNA and RNA. The heterodimerization at certain positions in a polynucleotide of two individual ZFNs in certain orientation and spacing can lead to cleavage of the polynucleotide. For example, a ZFN binding to DNA can induce a double-strand break in the DNA. In order to allow two cleavage domains to dimerize and cleave DNA, two individual ZFNs can bind opposite strands of DNA with their C-termini at a certain distance apart. In some cases, linker sequences between the zinc finger domain and the cleavage domain can require the 5′ edge of each binding site to be separated by about 5-7 base pairs. In some cases, a cleavage domain is fused to the C-terminus of each zinc finger domain. Exemplary ZFNs include, but are not limited to, those described in Urnov et al., Nature Reviews Genetics, 2010, 11:636-646; Gaj et al., Nat Methods, 2012, 9(8):805-7; U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; and U.S. Publication Nos. 2003/0232410 and 2009/0203140.

In some embodiments, a nuclease comprising a ZFN can generate a double-strand break in a target polynucleotide, such as DNA. A double-strand break in DNA can result in DNA break repair which allows for the introduction of gene modification(s) (e.g., nucleic acid editing). DNA break repair can occur via non-homologous end joining (NHEJ) or homology-directed repair (HR). In HR, a donor DNA repair template or template polynucleotide that contains homology arms flanking sites of the target DNA can be provided. In some embodiments, a ZFN is a zinc finger nickase which induces site-specific single-strand DNA breaks or nicks, thus resulting in HR. Descriptions of zinc finger nickases are found, e.g., in Ramirez et al., Nuel Acids Res, 2012, 40(12):5560-8; Kim et al., Genome Res, 2012, 22(7):1327-33. In some embodiments, a ZFN binds a polynucleotide (e.g., DNA and/or RNA) but is unable to cleave the polynucleotide.

In some embodiments, the cleavage domain of a nuclease comprising a ZFN comprises a modified form of a wild-type cleavage domain. The modified form of the cleavage domain can comprise an amino acid change (e.g., deletion, insertion, or substitution that reduces the nucleic acid-cleaving activity of the cleavage domain. For example, the modified form of the cleavage domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type cleavage domain. The modified form of the cleavage domain can have no substantial nucleic acid-cleaving activity. In some embodiments, the cleavage domain is enzymatically inactive.

In some embodiments, a nuclease suitable for use in the systems or methods described herein is a “TALEN” or “TAL-effector nuclease.” TALENs refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated Fok1 endonuclease or the catalytic domain of Fok1. Several mutations to Fok1 have been made for its use in TALENs, which, for example, improve cleavage specificity or activity. Such TALENs can be engineered to bind any desired DNA sequence. TALENs can be used to generate gene modifications (e.g., nucleic acid sequence editing) by creating a double-strand break in a target DNA sequence, which in turn, undergoes NHEJ or HR. A double-strand break in DNA can result in DNA, break repair which allows for the introduction of gene modification(s) (e.g., nucleic acid editing). DNA break repair can occur via non-homologous end joining (NHEJ) or homology-directed repair (HR). In HR, a donor DNA repair template or template polynucleotide that contains homology arms flanking sites of the target DNA can be provided. In some cases, a single-stranded donor DNA repair template is provided to promote HR. Detailed descriptions of TALENs and their uses for gene editing are found, e.g., in U.S. Pat. Nos. 8,440,431; 8,440,432; 8,450,471; 8,586,363; and 8,697,853; Scharenherg et al,, Curr Gene Ther, 2013, 13(4):291-303; Gaj et al., Nat Methods, 2012, 9(8):805-7; Beurdeley et al,, Nat Commun, 2013, 4:1762; and Joung and Sander, Nat Rev Mol Cell Biol, 2013, 14(1):49-55.

In some embodiments, a TALEN is engineered for reduced nuclease activity. In some embodiments, the nuclease domain of a TALEN comprises a modified form of a wild-type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive.

In some embodiments, the transcription activator-like effector (TALE) protein is fused to a domain that can modulate transcription and does not comprise a nuclease. In some embodiments, the transcription activator-like effector (TALE) protein is designed to function as a transcriptional activator. In some embodiments, the transcription activator-like effector (TALE) protein is designed to function as a transcriptional repressor. For example, the DNA-binding domain of the transcription activator-like effector (TALE) protein can be fused (e.g., linked) to one or more transcriptional activation domains, or to one or more transcriptional repression domains. Non-limiting examples of a transcriptional activation domain include a herpes simplex VP16 activation domain and a tetrameric repeat of the VP16 activation domain, e.g., a VP64 activation domain. A non-limiting example of a transcriptional repression domain includes a Kruppel-associated box domain.

In some embodiments, a nuclease suitable for use in the systems or methods described herein is a meganuclease. Meganucleases generally refer to rare-cutting endonucleases or horning endonucleases that can be highly specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. A meganuclease can generate a double-stranded break. A double-strand break in DNA can result in DNA break repair which allows for the introduction of gene modification(s) (e.g., nucleic acid editing). DNA break repair can occur via non-homologous end joining (NHEJ) or homology-directed repair (HR). In HR, a donor DNA repair template or template polynucleotide that contains homology arms flanking sites of the target DNA can be provided. The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-Msol meganuclease, I-SceI meganuclease, variants thereof, derivatives thereof, and fragments thereof. Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Nati Acad Sci USA, 2014, 111(11):4061-4066, and U.S. Pat. Nos. 7,842,489; 7,897,372; 8,021,867; 8,163,514; 8,133,697, 8,021,867; 8,119,361; 8,119,381; 8,124,36; and 8,129,134.

In some embodiments, the nuclease domain of a meganuclease comprises a modified form of a wild-type nuclease domain. The modified form of the nuclease domain can comprise an amino acid change (e.g., deletion, insertion, or substitution) that reduces the nucleic acid-cleaving activity of the nuclease domain. For example, the modified form of the nuclease domain can have no more than 90%, no more than 80%, no more than 70%, no more than 60%, no more than 50%, no more than 40%, no more than 30%, no more than 20%, no more than 10%, no more than 5%, or no more than 1% of the nucleic acid-cleaving activity of the wild-type nuclease domain. The modified form of the nuclease domain can have no substantial nucleic acid-cleaving activity. In some embodiments, the nuclease domain is enzymatically inactive. In some embodiments, a meganuclease can bind DNA but cannot cleave the DNA.

In some embodiments, the nuclease is fused to one or more transcription repressor domains, activator domains, epigenetic domains, recombinase domains, transposase domains, flippase domains, nickase domains, or any combination thereof. The activator domain can include one or more tandem activation domains located at the carboxyl terminus of the enzyme. In other cases, the actuator moiety includes one or more tandem repressor domains located at the carboxyl terminus of the protein. Non-limiting exemplary activation domains include GAL4, herpes simplex activation domain VP16, VP64 (a tetramer of the herpes simplex activation domain VP16), NF-KB p65 subunit, Epstein-Barr virus R transactivator (Rta) and are described in Chavez et al., Nat Methods, 2015, 12(4):326-328 and U.S. Patent App. Publ. No. 20140068797. Non-limiting exemplary repression domains include the KRAB (Kruppel-associated box) domain of Koxl, the Mad mSIN3 interaction domain (SID), ERF repressor domain (ERD), and are described in Chavez et al., Nat Methods, 2015, 12(4):326-328 and U.S. Patent App. Publ. No. 20140068797. A nuclease can also be fused to a heterologous polypeptide providing increased or decreased stability. The fused domain or heterologous polypeptide can be located at the N-terminus, the C-terminus, or internally within the nuclease.

A nuclease can comprise a heterologous polypeptide for case of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (e.g., mOrange, mKO, Kusabira-Orange, Monomeric Kusabira-Orange, mTangerine, tdTornato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, hemagglutinin (HA), nus, Softag 1, Softag 3, Strep, SBP, Glu-Glu, HSV, KT3, S, SI, T7, V5, VSV-G, histidine (His), biotin carboxyl carrier protein (BCCP), and calmodulin.

In some embodiments, the nuclease and the second dimerization domain are linked via a linker. A linker can be any linker known in the art. In some embodiments, the nuclease and second dimerization domain are linked as fusion protein.

### D. Guide Nucleic Acids

In some cases, the systems and methods described herein comprise at least one guide nucleic acid polynucleotide. In some cases, the systems and methods described herein comprise a plurality of guide nucleic acids. In some embodiments, the polynucleotide can be deoxyribonucleic acid (DNA). In some cases, the DNA sequence can be single-stranded or doubled-stranded. In a preferred embodiment, the at least one guide nucleic acid polynucleotide can be ribonucleic acid (guide RNA).

In some embodiments, the nuclease can be complexed with the at least one guide RNA polynucleotide. The at least one guide RNA polynucleotide can comprise a nucleic-acid targeting region that comprises a complementary sequence to a nucleic acid sequence on the targeted polynucleotide such as the targeted mammalian genomic loci, mammalian genes, human genomic loci, or human genes to confer sequence specificity of nuclease targeting. In some embodiments, the at least one guide RNA polynucleotide can comprise two separate nucleic acid molecules, which can be referred to as a double guide nucleic acid or a single nucleic acid molecule, which can be referred to as a single guide nucleic acid (e.g., single guide RNA or sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracrRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like molecule and a tracrRNA-like molecule.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used.

In some embodiments, the nucleic acid-targeting region of a guide nucleic acid can be between 18 to 72 nucleotides in length. The nucleic acid-targeting region of a guide nucleic acid (e.g., spacer region) can have a length of from about 12 nucleotides to about 100 nucleotides. For example, the nucleic acid-targeting region of a guide nucleic acid (e.g., spacer region) can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, from about 12 nt to about 19 nt, from about 12 nt to about 18 nt, from about 12 nt to about 17 nt, from about 12 nt to about 16 nt, or from about 12 nt to about 15 nt. Alternatively, the DNA-targeting segment can have a length of from about 18 nt to about 20 nt, from about 18 nt to about 25 nt, from about 18 nt to about 30 nt, from about 18 nt to about 35 nt, from about 18 nt to about 40 nt, from about 18 nt to about 45 nt, from about 18 nt to about 50 nt, from about 18 nt to about 60 nt, from about 18 nt to about 70 nt, from about 18 nt to about 80 nt, from about 18 nt to about 90 nt, from about 18 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The length of the nucleic acid-targeting region can be at least 5, 10, 15, 16, 17, 18, 19, 20, 21, 22, 23, 24, 25, 30 or more nucleotides. The length of the nucleic acid-targeting region (e.g., spacer sequence) can be at most 5, 10, 15, 16, 17, 18, 19, 20, 21, 22, 23, 24, 25, 30 or more nucleotides.

In some embodiments, the nucleic acid-targeting region of a guide nucleic acid (e.g., spacer) is 20 nucleotides in length. In some embodiments, the nucleic acid-targeting region of a guide nucleic acid is 19 nucleotides in length. In some embodiments, the nucleic acid-targeting region of a guide nucleic acid is 18 nucleotides in length. In some embodiments, the nucleic acid-targeting region of a guide nucleic acid is 17 nucleotides in length. In some embodiments, the nucleic acid-targeting region of a guide nucleic acid is 16 nucleotides in length. In some embodiments, the nucleic acid-targeting region of a guide nucleic acid is 21 nucleotides in length. In some embodiments, the nucleic acid-targeting region of a guide nucleic acid is 22 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt.

A protospacer sequence of a targeted polynucleotide can be identified by identifying a protospacer-adjacent motif (PAM) within a region of interest and selecting a region of a desired size upstream or downstream of the PAM as the protospacer. A corresponding spacer sequence can be designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility, % GC, frequency of genomic occurrence, methylation status, presence of SNPs, and the like.

The percent complementarity between the nucleic acid-targeting sequence (e.g., a spacer sequence of the at least one guide polynucleotide as disclosed herein) and the target nucleic acid (e.g., a protospacer sequence of the one or more target genes as disclosed herein) can be at least 50%, at least 60%, at least 70%, at least 75%, at least 80%, at least 85%, at least 90%, at least 95%, at least 97%, at least 98%, at least 99%, or 100%. The percent complementarity between the nucleic acid-targeting sequence and the target nucleic acid can be at least 60%, at least 70%, at least 75%, at least 80%, at least 85%, at least 90%, at least 95%, at least 97%, at least 98%, at least 99%, or 100% over about 20 contiguous nucleotides.

The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and crRNA can be covalently linked via the 5′ end of the tracrRNA and the 3′ end of the crRNA.

The Cas protein binding segment of a guide nucleic acid can have a length of from about 10 nucleotides to about 100 nucleotides, e.g., from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. For example, the Cas protein-binding segment of a guide nucleic acid can have a length of from about 15 nucleotides (nt) to about 80 nt, from about 15 nt to about 50 nt, from about 15 nt to about 40 nt, from about 15 nt to about 30 nt or from about 15 nt to about 25 nt.

The dsRNA duplex of the Cas protein-binding segment of the guide nucleic acid can have a length from about 6 base pairs (bp) to about 50 bp. For example, the dsRNA duplex of the protein-binding segment can have a length from about 6 bp to about 40 bp, from about 6 bp to about 30 bp, from about 6 bp to about 25 bp, from about 6 bp to about 20 bp, from about 6 bp to about 15 bp, from about 8 bp to about 40 bp, from about 8 bp to about 30 bp, from about 8 bp to about 25 bp, from about 8 bp to about 20 bp or from about 8 bp to about 15 bp. For example, the dsRNA duplex of the Cas protein-binding segment can have a length from about from about 8 bp to about 10 bp, from about 10 bp to about 15 bp, from about 15 bp to about 18 bp, from about 18 bp to about 20 bp, from about 20 bp to about 25 bp, from about 25 bp to about 30 bp, from about 30 bp to about 35 bp, from about 35 bp to about 40 bp, or from about 40 bp to about 50 bp.

In some embodiments, the dsRNA duplex of the Cas protein-binding segment can have a length of 36 base pairs. The percent complementarity between the nucleotide sequences that hybridize to form the dsRNA duplex of the protein-binding segment can be at least about 60%. For example, the percent complementarity between the nucleotide sequences that hybridize to farm the dsRNA duplex of the protein-binding segment can be at least about 65%, at least about 70%, at least about 75%, at least about 80%, at least about 85%, at least about 90%, at least about 95%, at least about 98%, or at least about 99%. In some cases, the percent complementarity between the nucleotide sequences that hybridize to form the dsRNA duplex of the protein-binding segment is 100%.

The linker (e.g., the sequence that links a crRNA and a tracrRNA in a single guide nucleic acid) can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a DNA-targeting RNA is 4 nt.

Guide nucleic acids of the disclosure can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (a 7-methylguanylate cap (m7G)); a 3′ polyadenylated tail (a 3′ poly(A) tail); a riboswitch sequence (e.g., to allow for regulated stability and/or regulated accessibility by proteins and/or protein complexes); a stability control sequence; a sequence that forms a dsRNA duplex (a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyl transferases, DNA demethylases, histone acetyltransferases, histone deacetylases, and combinations thereof.

A guide nucleic acid can comprise one or more modifications (e.g., a base modification, a backbone modification), to provide the nucleic acid with a new or enhanced feature (e.g., improved stability). A guide nucleic acid can comprise a nucleic acid affinity tag. A nucleoside can be a base-sugar combination. The base portion of the nucleotide can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming guide nucleic acids, the phosphate groups can covalently link adjacent nucleosides to one another to form a linear polymeric compound. In turn, the respective ends of this linear polymeric compound can be further joined to form a circular compound; however, linear compounds can be suitable. In addition, linear compounds can have internal nucleotide base complementarity and can therefore fold in a manner as to produce a fully or partially double-stranded compound. Further, within guide nucleic acids, the phosphate groups can commonly be referred to as forming the internucleoside backbone of the guide nucleic acid. The linkage or backbone of the guide nucleic acid can be a 3′ to 5′ phosphodiester linkage.

A guide nucleic acid can comprise a modified backbone and/or modified internucleoside linkages. Modified backbones can include those that retain a phosphorus atom in the backbone and those that do not have a phosphorus atom in the backbone.

Suitable modified guide nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoratnidate and aminoalkylphosphoramidates, phosphorodiamidates, thionophosphoramidates, thionoalkyiphosphonates, thionoalkylphosphotriesters, selenophosphates, and boranophosphates having normal 3′-5′ linkages, 2′-5′ linked analogs, and those having inverted polarity wherein one or more intemucleotide linkages is a 3′ to 3′, a 5′ to 5′ or a 2′ to 2′ linkage. Suitable guide nucleic acids having inverted polarity can comprise a single 3′ to 3′ linkage at the 3′-most internucleotide linkage (such as a single inverted nucleoside residue in which the nucleobase is missing or has a hydroxyl group in place thereof). Various salts (e.g., potassium chloride or sodium chloride), mixed salts, and free acid forms can also be included.

A guide nucleic acid can comprise one or more phosphorothioate and/or heteroatom internucleoside linkages, in particular —CH2-NH—O—CH2-, —CH2-N(CH3)-O—CH2- (a methylene nicthylimino) or MMI backbone), —CH2-O—N(CH3)-CH2-, —CH2-N(CH3)-N(CH3)-CH2- and —O—N(CH3)-CH2-CH2- (wherein the native phosphodiester intemucleotide linkage is represented as —O—P(═O)(OH)—O—CH2-).

A guide nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage replaces a phosphodiester linkage.

A guide nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacctyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

A guide nucleic acid can comprise a nucleic acid mimetic. The term “mimetic” can be intended to include polynucleotides wherein only the furanose ring or both the furanose ring and the intemucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A guide nucleic acid can comprise linked morpholino units morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be non-ionic mimics of guide nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C). stability towards 3′-exonucleolytic degradation and good solubility properties.

A guide nucleic acid can comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. A sugar substituent group can be selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an guide nucleic acid, or a group for improving the pharmacodynamic properties of an guide nucleic acid, and other substituents having similar properties. A suitable modification can include 2′-methoxyethoxy (2′-O—CH₂ CH₂OCH₃, also known as 2′-O-(2-methoxyethyl) or 2′-MOE, an alkoxyalkoxy group). A further suitable modification can include 2′-dimethylaminooxyethoxy, (a O(CH₂)₂ON(CH₃)₂ group, also known as 2′-DMAOE), and 2′-dimethylaminoethoxyethoxy (also known as 2′-O-dimethyl-amino-ethoxy-ethyl or 2′-DMAEOE), 2′-O—CH₂—O—CH₂—N(CH₃)₂.

Other suitable sugar substituent groups can include methoxy (—O—CH₃), aminopropoxy (—O CH₂ CH₂NH₂), allyl (—CH₂—CH₂═CH₂), —O-allyl (—O—CH₂—CH═CH₂) and fluoro (F). 2′-sugar substituent groups can be in the arabino (up) position or ribo (down) position. A suitable 2′-arabino modification is 2′-F. Similar modifications can also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds can also have sugar mimetics such as cyclobutyl moieties in place of the pentofuranosyl sugar.

A guide nucleic acid can also include nucleobase (or “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8.-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino¬adenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H¬pyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of a guide nucleic acid can comprise chemically linking to the guide nucleic acid one or more moieties or conjugates that can enhance the activity, cellular distribution or cellular uptake of the guide nucleic acid. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octa.decylamine or hexylamino-carbonyl-oxycholesterol moiety.

In some embodiments, the at least one guide RNA polynucleotide can bind to at least a portion of the mammalian genomes, mammalian genes, human genomes, or human genes. In some cases, the at least one guide RNA polynucleotide is capable of forming a complex with the nuclease to direct the nuclease to target the portion of the mammalian genomes, mammalian genes, human genomes, or human genes.

In some embodiments, the at least one guide RNA polynucleotide can be complementary and bind to the mammalian genomes, mammalian genes, human genomes, or human genes as described herein.

In some embodiments, the systems and methods described herein comprise complexing the at least one guide RNA polynucleotide with the nuclease. In some embodiments, the systems and methods described herein comprise complexing at least two different guide RNA polynucleotides with the nuclease. In some embodiments, the systems and methods described herein comprise complexing at least three different guide RNA polynucleotides with the nuclease. In some embodiments, the systems and methods described herein comprise complexing at least four different guide RNA polynucleotides with the nuclease. In some embodiments, the systems and methods described herein comprise complexing at least five different guide RNA polynucleotides with the nuclease. In some embodiments, the systems and methods described herein comprise complexing at least six different guide RNA polynucleotides with the nuclease.

### E. Delivery

Described herein, in some embodiments, are methods of targeting integration of at least one payload into at least one genomic locus in a host cell, preferably a mammalian cell. In some embodiments, the methods comprise introducing at least a first nuclease targeted to at least one genomic locus into a host cell. In some embodiments, the methods comprise introducing a donor template or vector comprising at least one payload into a host cell. In some embodiments, the methods comprise introducing both a first nuclease targeted to at least one genomic locus and a donor template or vector comprising at least one payload into a host cell. The nuclease and the donor template or vector can be introduced into the host cell by well-known methods, which vary depending on the type of host cell.

As used herein, the phrase “introducing” in the context of introducing a polypeptide (e.g., a nuclease) into a cell refers to the delivery or translocation of either the polypeptide itself or a nucleic acid encoding the polypeptide from outside a cell to inside the cell. In some embodiments, the polypeptide may be directly delivered to the cell by known methods, including liposome-mediated transfection or electroporation. For example, delivery of a ribonucleoprotein (RNP) complex containing a Cas protein complexed with a guide nucleic acid (e.g., a guide RNA) targeting the desired locus may be performed by liposome-mediated transfection or electroporation. In some embodiments, the modified host cell is in contact with a medium containing serum following electroporation. In some embodiments, the modified host cell is in contact with a medium containing reduced serum or containing no serum following electroporation. In some embodiments, the polypeptide is delivered to the cell via introduction of a nucleic acid encoding the polypeptide.

As used herein, the phrase “introducing” in the context of introducing a nucleic acid (e.g., a donor template or vector) into a cell refers to the translocation of nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanopaiticle delivery, viral delivery, contact with nanowires or nanotubes, receptor mediated internalization, translocation via cell penetrating peptides, liposome mediated translocation, DEAF dextran, lipofectamine, calcium phosphate or any method now known or identified in the future for introduction of nucleic acids into prokaryotic or eukaryotic cellular hosts. A targeted nuclease system (e.g., an RNA-guided nuclease (CRISPR-Cas9), a transcription activator-like effector nuclease (TALEN), a zinc finger nuclease (ZFN), or a megaTAL (MT) (Li et al. Signal Transduction and Targeted Therapy 5, Article No. 1 (2020)) can also be used to introduce a nucleic acid, for example, a nucleic acid encoding a recombinant protein described herein, into a host cell.

In some embodiments, the nuclease and the guide RNA polynucleotide can be delivered into the cell. In some embodiments, polynucleotides encoding the nuclease and/or the guide RNA polynucleotide can be delivered into the cell via the use of expression vectors. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, gene gun, electroporation, and the like. Methods for producing cells comprising vectors and/or exogenous nucleic acids are suitable for methods herein (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors, in some embodiments, are derived from lentivirus, pseudoviruses, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted, nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape, Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. For example, in some embodiments, dimyristyl phosphatidylcholine (“DMPC”) is obtained from Sigma, St. Louis, Mo.; in some embodiments, dicetyl phosphate (“DCP”) is obtained from K & K Laboratories (Plainview, N.Y.); cholesterol (“Choi”), in some embodiments, is obtained from Calbiochem-Behring; dimyristyl phosphatidylglycerol (“DMPG”) and other lipids are often obtained from Avanti Polar Lipids, Inc. (Birmingham, Ala.). Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, the compositions described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

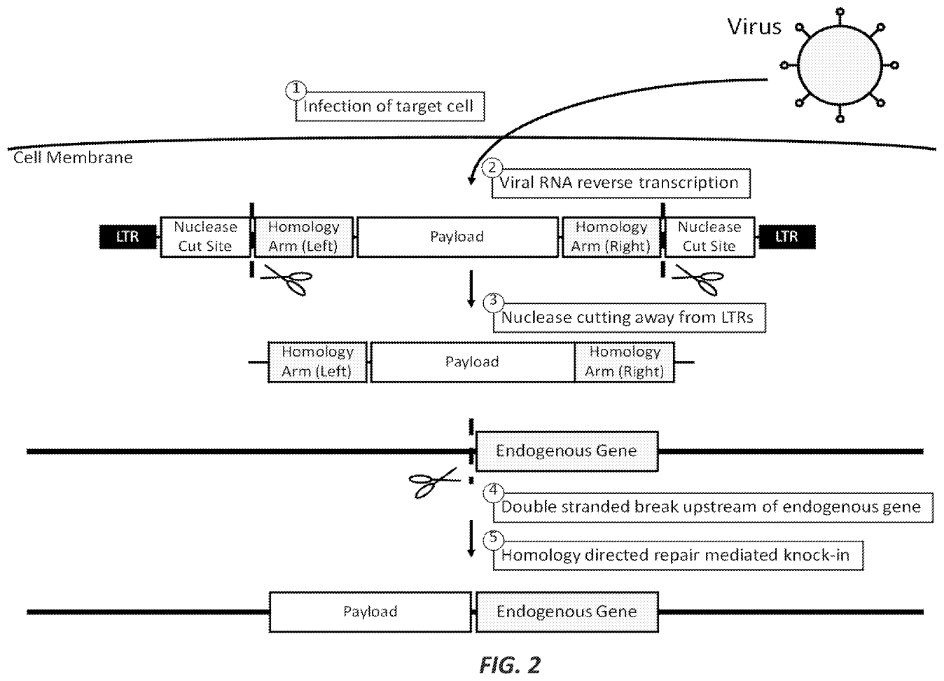

In some embodiments, the nuclease and the donor template or vector can be introduced into the host cell in two steps. In some embodiments, the donor template or vector is introduced at least 8 hours prior to the nuclease. For example, host cells may be transduced with pseudovirus particles (e.g., integration deficient lentivirus particles) comprising the donor template at a high multiplicity of infection (MOI; e.g., at least 50 or at least 100 plaque forming units per cell). In some embodiments, transduced pseudovirus particles release their RNA genome, reverse transcribe the genome into complementary DNA (cDNA), and amplify the cDNA copy number via repeated reverse transcription and replication (FIG. 2). In some embodiments, this amplification leads to a high donor template copy number. In some embodiments, the nuclease system is introduced to the host cells 8-72 hours (e.g., 12 hours, 16 hours, 20 hours, 24 hours, 36 hours, or 48 hours) after donor template introduction. For example, nuclease or nucleic acids encoding the nuclease and, optionally, guide nucleic acids to target the nuclease to a genomic locus (e.g., guide RNA) or nucleic acids encoding the guide nucleic acids may be delivered to the cell through any of the methods described above 12-48 hours after introduction of the donor template or vector.

In some embodiments, the nuclease and the donor template or vector can be introduced into the host cell in one step. In one embodiment, host cells may be transduced with pseudovirus particles (e.g., integration deficient lentivirus particles) comprising a donor template and nucleotide sequences encoding a nuclease and, optionally, a guide nucleic acid to target the nuclease to a genomic locus (e.g., guide RNA). In some embodiments, the nucleotide sequences encoding the nuclease and optional guide nucleic acid are packaged in the pseudovirus as a single RNA molecule or individual RNA molecules (e.g., not integrated into the viral genome). In some embodiments, a drug-inducible system may control nuclease expression or activity (e.g., through use of a small molecule inducible promoter such as TRE3G). In some embodiments, the nucleotide sequences encoding the nuclease and optional guide nucleic acid are incorporated into the viral genome along with the donor template. In some embodiments, the pseudovirus comprises the nuclease in protein form (e.g., packaged into the pseudovirus core or carried on the pseudovirus outer membrane).

In some embodiments, the nuclease (e.g., Cas9, Cas12, Cas14, or engineered versions thereof) may contain at least one copy of a nuclear localization signal intended to enhance transport to the nucleus. A nuclease which is more effectively transported to the nucleus may cleave host cell genomic DNA at the desired locus more efficiently. Further, as described above, the donor template or vector disclosed herein may comprise cleavage sites which can be bound or cleaved by a nuclease. In such instances, a nuclease which comprises at least one copy of a nuclear localization signal may also enhance transport of the donor template or vector to the nucleus through binding between the nuclease and the cleavage site.

### IV. Methods of Using Modified Cells

Also disclosed herein are methods of using the modified cells and/or vaccines described above in treatment of a subject. The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a modified cell or vaccine disclosed herein in a therapeutically effective amount. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the quantity of a composition, for example a composition comprising modified cells such as lymphocytes (e.g., T lymphocytes and/or NK cells) modified according to the methods of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

In one embodiment, provided herein is a method of inducing an immune response in a subject comprising administering the modified cells or vaccines of the present disclosure (e.g., by infusing the modified mammalian cell into the subject). In one embodiment, modified cells expressing an antigen from a human virus are administered to a subject to induce an immune response. In one embodiment, modified cells expressing the Spike protein or RNA dependent RNA polymerase protein from human SARS-CoV-2 are administered to a subject to induce an immune response. Such an immune response may provide a prophylactic benefit against a coronavirus, e.g. SARS-CoV-2.

### V. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise—Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “plurality” refers to more than one entity. Thus, a “plurality of individuals” refers to at least two individuals. A plurality may be 2, 3, 4, 5, 6, 7, 8, 9, 10, or more individuals within a larger population. Additionally, a plurality may be represented by 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98%, or 99% of the population.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides described herein also encompass all forms of sequences including, but not limited to, single-stranded forms, double-stranded forms, hairpins, stem-and-loop structures, and the like.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

The terms “complement,” “complements,” “complementary,” “complementarity,” and “percent complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%. 45%, 50%, 55%, 60%, 65%, 70%, 75%, 80%, 85%, 90%, 91%, 92%, 93%, 94%, 95%, 96%, 97%, 98%, 99%, and 100% sequence complementarity.

Complementarily can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

For sequence comparison, such as for the purpose of assessing sequence identity or complementarity, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Nati, Acad, Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as fax as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

As used throughout, the term “vector” refers to a nucleic acid molecule that is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as plasmid and viral vectors.

The term “antigen,” as used herein, refers to a molecule or a fragment thereof capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen.

Coronaviruses are a group of enveloped, single-stranded RNA viruses that cause diseases in mammals and birds. Coronavirus hosts include bats, pigs, dogs, cats, mice, rats, cows, rabbits, chickens and turkeys. In humans, coronaviruses cause mild to severe respiratory tract infections. Coronaviruses vary significantly in risk factor. Some can kill more than 30% of infected subjects. The following strains of human coronaviruses are currently known: Human coronavirus 229E (HCoV-229E); Human coronavirus OC43 (HCoV-OC43), Severe acute respiratory syndrome coronavirus (SARS-CoV or SARS-CoV-1); Human coronavirus NL63 (HCoV-NL63, New Haven coronavirus); Human coronavirus HKU1 (HCoV-HKU1), which originated from infected mice, was first discovered in January 2005 in two patients in Hong Kong; Middle East respiratory syndrome-related coronavirus (MERS-CoV), also known as novel coronavirus 2012 and HCoV-EMC; and Severe acute respiratory syndrome coronavirus 2 (SARS-COV-2), also known as 2019-nCoV or “novel coronavirus 2019.” The coronaviruses HCoV-229E, -NL63, -OC43, and -HKU1 continually circulate in the human population and cause respiratory infections in adults and children world-wide.

A “Spike” protein is one of a group of coronavirus surface proteins that are able to mediate receptor binding and membrane fusion between the virus and host cell. Spikes are homotrimers of the S protein, which has S1 and S2 domains. In addition to mediating virus entry, the spike is an important determinant of viral host range and tissue tropism and a major inducer of host immune responses. The S1 subunit of the S protein includes the receptor binding domain (RBD).

### EXAMPLES

The following examples are offered to illustrate, but not to limit the claimed invention.

### Example 1. Methods

Design of Donors. Cas9 guides that cut at a specific point around a genomic region of interest were designed in silico, introduced into HEK293T with Cas9 via plasmid transfection, and assayed for their ability to cut via TIDE-seq of PCR fragments from isolated gDNA. After a cut-site was found, homology arms were amplified from genomic DNA via PCR. This amplification introduced the 20 base pair+PAM sequence that allow for targeted cutting of the donor. Desired payload is amplified from parts in the Qi lab library. The two homology arms and the payload are cloned into a lentiviral pHR vector.

Cancer Cell Line Knock-in. Integrase deficient lentivirus (IDLV) was created using standard protocols that included transfecting the pHR vector (described above), the pCMV-R8.91 vector containing the integrase deficient D64V mutation, and the pMD.2 vector into HEK293T cells. Three days later, virus was isolated by filtration of culture supernatant. Virus was then concentrated by centrifugation and titered by qPCR.

Cancer cell lines (K562, EL4, and Jurkat) were seeded at a density of 100,000 cells per well of a 96 well plate. IDLV was added at a multiplicity of infection (MOI) of 1000 and allowed to incubate for 24 hours. Cells were then pelleted and resuspended in 20 μL of SF, SG, or SE Cell Line Nucleofector Solution for a final concentration of 10⁷ cells/mL, according to Lonza optimized protocols. Cas9 ribonucleoprotein (RNP) was created by incubating 50 pmol of Cas9 protein with 100 pmol modified Synthego sgRNAs for 10 min in PBS at room temperature. RNP and cells were mixed and added to a Lonza 4D nucleocuvette. Nucleofection was performed per Lonza protocol. Cells were assayed by flow cytometry 7 days after nucleofection.

Primary T cell Knock-in. IDLV was made as described above. Prior to this, primary CD3+ T cells were isolated from buffy coat of patient samples and cryopreserved. On Day 1, primary T cells were thawed and incubated at 1,000,000 cells/mL, in 200 U/mL, of IL2, 5 ng/mL IL7, and 5 ng/mL IL15, and 10⁶ beads/mL CD3/CD28 Dynaheads. On Day 2, IDLV was added at an MOI of 1000 and incubated for 24 hours. On Day 3, beads were removed and 1,000,000 infected cells were pelleted and resuspended in 20 μL P3 Primary Cell Nucleofector Solution for a final concentration of 5×10⁷ cells/mL. 130 pmol RNP was created as described above and mixed with cells. Cells were nucleofected using Lonza protocols and resuspended in high IL2 (500 U/mL) media. Cells were assayed by flow cytometry 7 days after nucleofection.

### Example 2. Addition of Nuclease Cut Sites to Viral Donor Improves Knock-In Efficiency

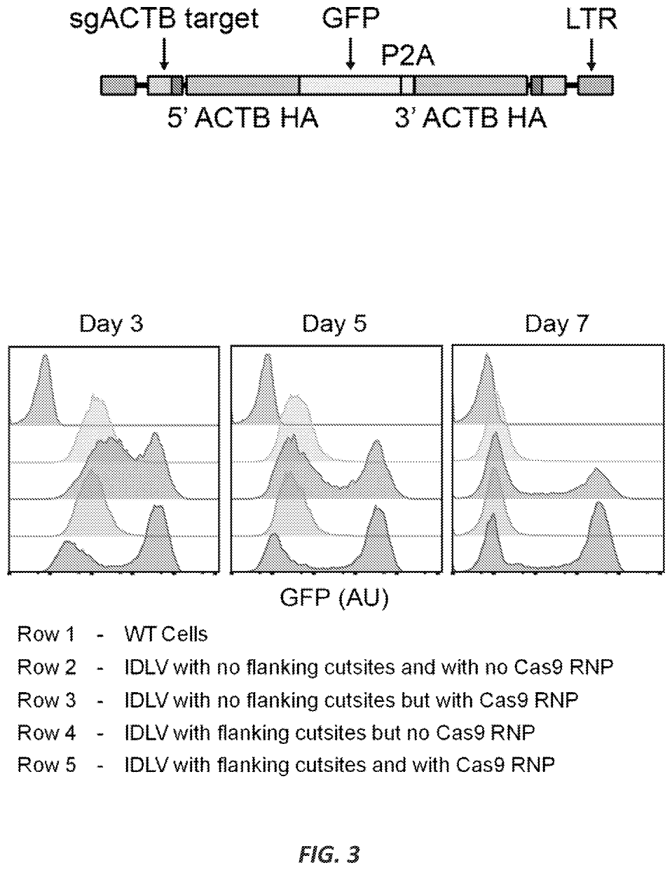

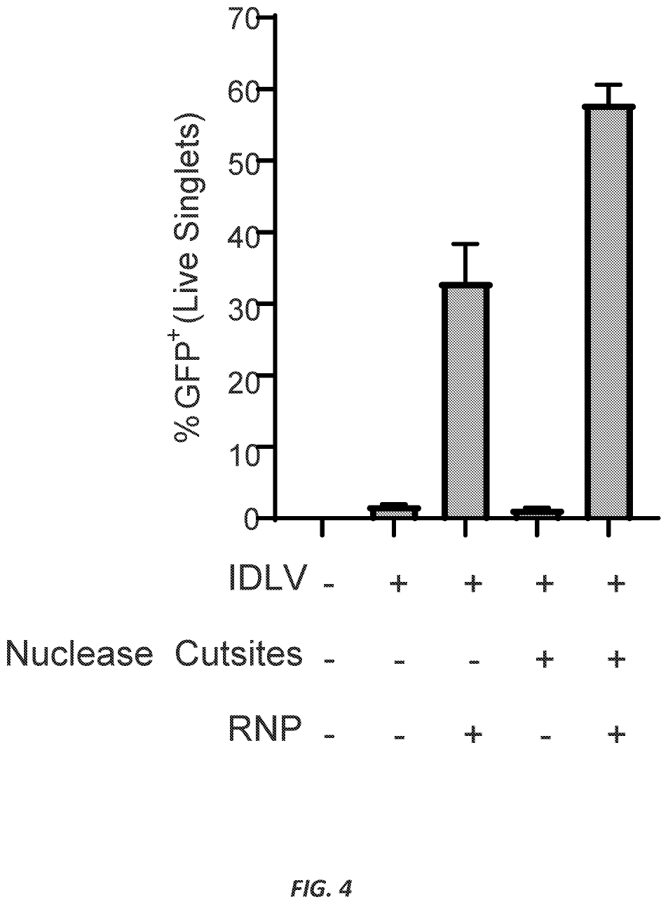

Donor templates were designed with a green fluorescent protein (GFP) gene payload and homology arms for ACTB with or without flanking cleavage sites matching the genomic cleavage site as shown in FIG. 3. K562 cells were infected with IDLV containing the donor templates as described in Example 1, incubated for 24 hours, and nucleofected with RNP containing Cas9 protein and sgRNA to target Cas9-mediated cleavage to the genomic cleavage site as described in Example 1. The cells were then incubated and assayed for GFP fluorescence using flow cytometry at days 3, 5, and 7, as shown in FIG. 3. As shown in FIG. 4, donor templates without cleavage sites flanking the homology arms were knocked in less efficiently than those with cleavage sites, as indicated by a higher percentage of GFP positive cells detected by flow cytometry.

### Example 3. Knock-In Method Efficiency Can be Predicted a Priori

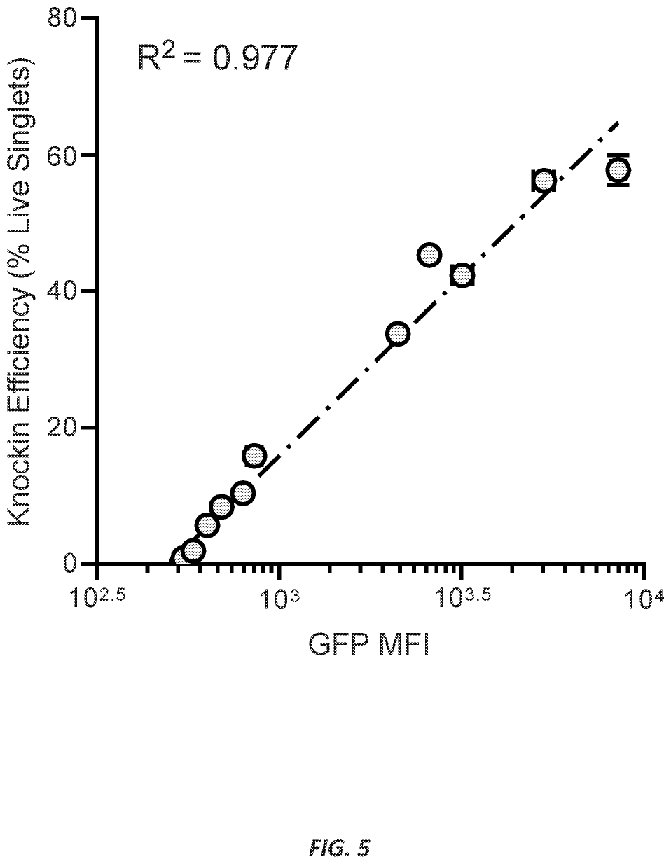

Donor templates were designed with a green fluorescent protein (GFP) gene payload and homology arms for ACTB with flanking cleavage sites matching the genomic cleavage site as above. K562 cells were infected with IDLV containing the donor templates as described in Example 1 at various concentrations. Before addition of Cas9 RNP, transduction led to GFP expression from the lentiviral genome, as seen in FIG. 3. This was assayed 24 hours after transduction by flow cytometry, before RNP nucleofection. Cells were then nucleofected with Cas9-RNP targeting ACTB and assayed by flow cytometry 7 days later. As seen in FIG. 5, the magnitude of expression before nucleofection had high correlation to the knock-in efficiency, suggesting efficiency could be predicted before the knock-in was performed.

### Example 4. Knock-In Method is Effective at Multiple Genomic Loci

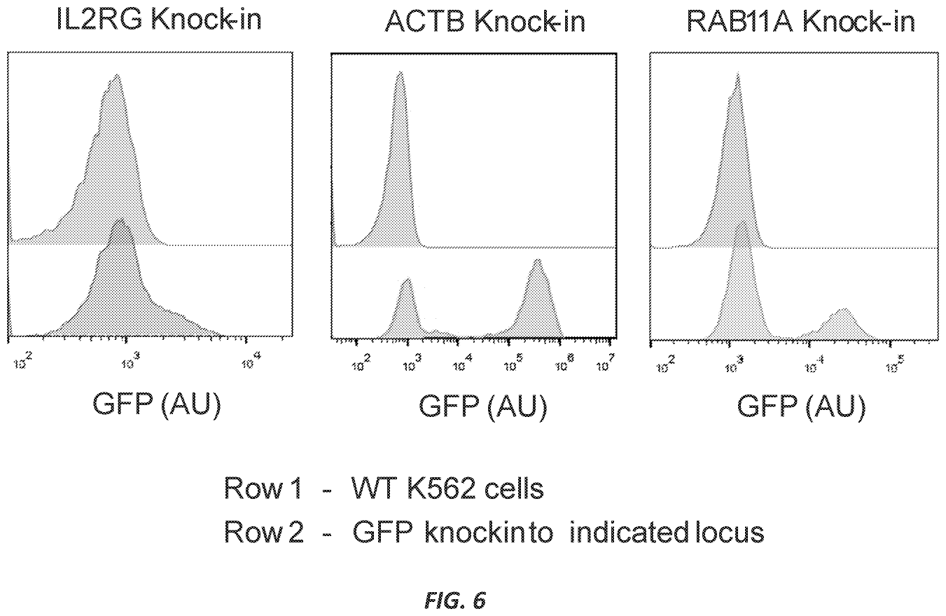

To confirm that the knock-in methods described herein are able to target payloads to various genomic loci, donor templates with fluorescent gene payloads were designed and targeted upstream of IL2RG, upstream of ACTB, and upstream of RAB11A. All templates included homology arms to their target as well as flanking cleavage sites corresponding to the genomic cleavage sites. Fluorescent protein expression was assayed by flow cytometry. As shown in FIG. 6, payload integration was successful at all three locations, as indicated by increased fluorescence.

### Example 5. Knock-In Method Enables Targeted Integration of Large Payloads

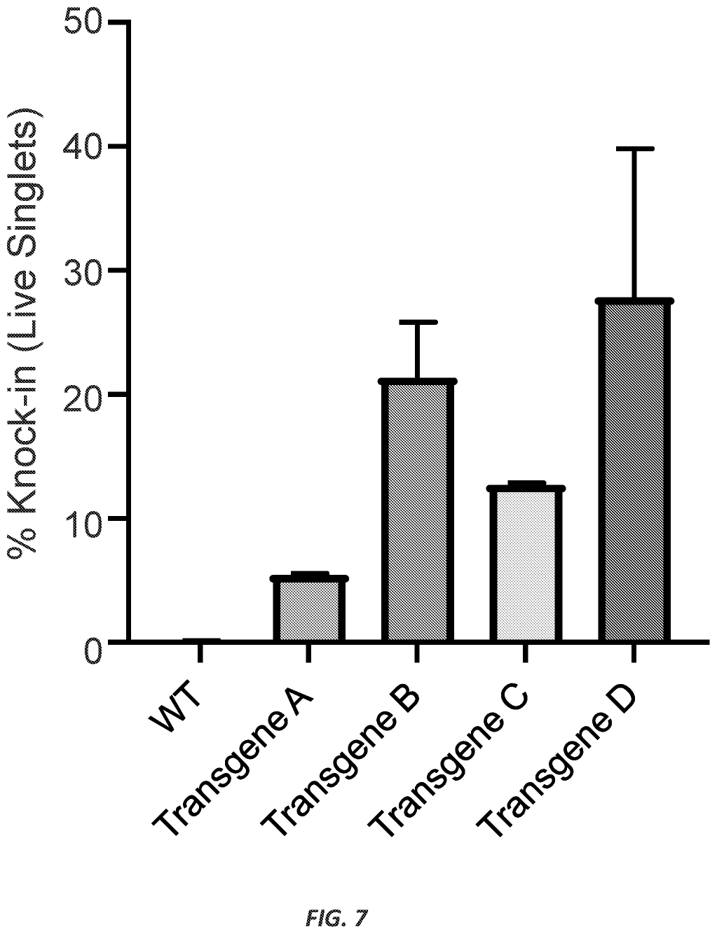

The knock-in methods described herein were used to insert several transgenes from toxic sources, which are large (greater than 3 kb) and hard to express, into the ACTB locus in Jurkat cells, as described in Example 1. As shown in FIG. 7, three different large transgenes were successfully inserted into the locus, as indicated by the presence of GTP positive cells measured by flow cytometry. Transgene A is the toxic S1 region of the SARS CoV-2 Spike protein fused to GFP (3.7 kb total). Transgene B is the SARS-CoV-2 RNA dependent RNA polymerase (RdRP) fused to GFP (3.6 kb total). Transgene C is the toxic S1 region, the RdRP, and GFP (5.7 kb total). Transgene D, which is GFP alone (0.7 kb), is included for comparison.

### Example 6. Knock-In of Multiple Transgenes at Multiple Genomic Loci Can be Performed Using a Single Vector

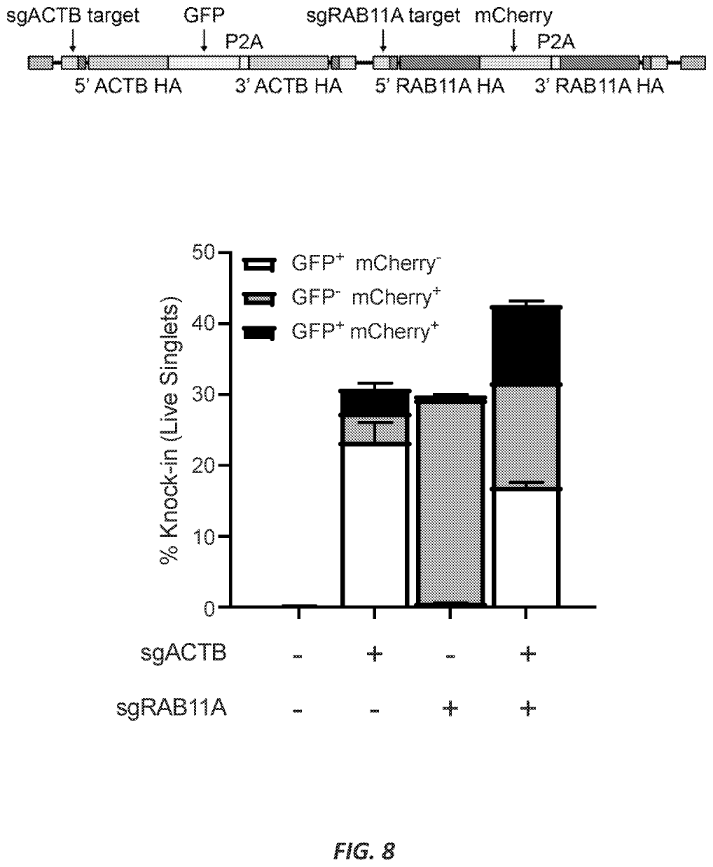

The large payload capacity of lentiviral vectors along with inclusion of cleavage sites flanking the homology arms, as described herein, allows for multiple knock-ins using one viral vector. As shown in FIG. 8, a single IDLV containing two donor templates, one with a GFP transgene payload and homology arms for the ACTB locus flanked by the ACTB cleavage site, and one with an mCherry fluorescent protein transgene payload and homology arms for the RAB11A locus flanked by the RAB11A cleavage site, was transduced into K562 cells as described above. Here, 100,000 K562s cells were transduced with the IDLV described at an MOI of 1000 and incubated for 24 hours. Cells were resuspended in SF Cell Line Nucleofection Solution as described in Example 1. Two Cas9 RNPs were created by separately mixing 50 pmol of Cas9 HiFi with either 100 pmol sgRNA targeting RAB11A or ACTB. Cells were mixed with both RNPs and electroporated as in Example 1. As shown in FIG. 8, introduction of Cas9 with sgRNAs targeting it to the ACTB cleavage site alone or to the RAB111A cleavage site alone led to a high percentage of cells expressing only the fluorescent reporter targeted to the respective locus. However, introduction of C7as9 with sgRNAs targeting it to both the ACTB cleavage site and the RAB11A cleavage site led to a high percentage of cells expressing both fluorescent reporters.

### Example 7. Knock-In Method Integrates Transgenes into Multiple Essential Loci in Primary Cells

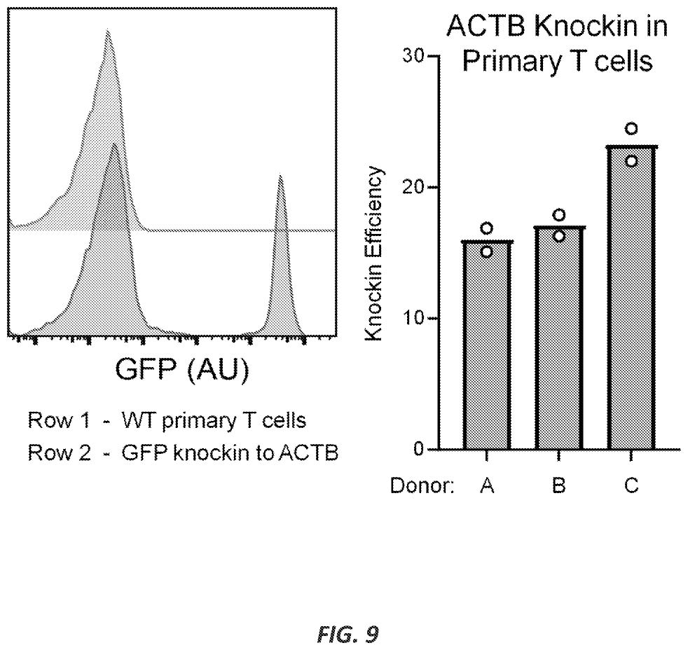

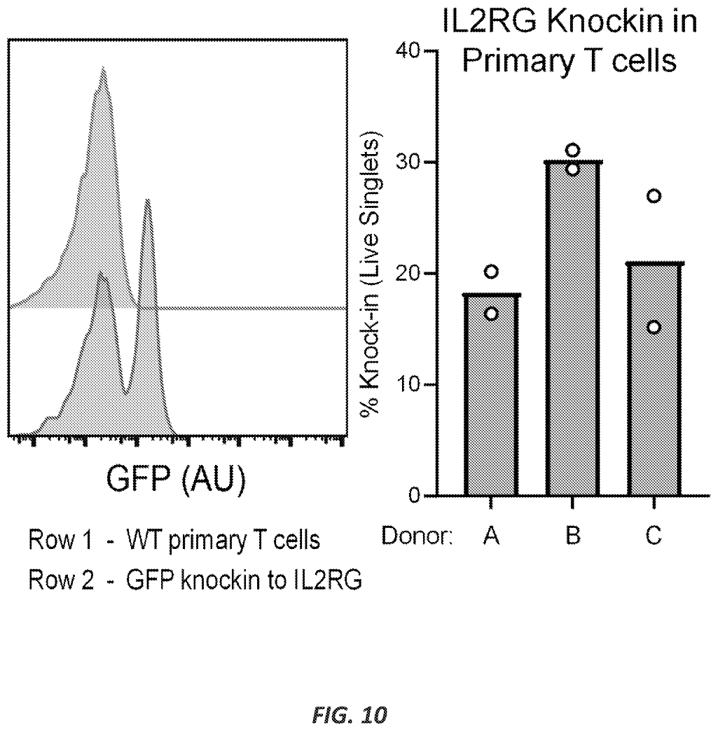

A knock-in strategy as described herein was also tested in primary T cells, a therapeutically important cell type, as described in Example 1. As shown in FIG. 9 and FIG. 10, fluorescent transgene payloads were successfully inserted into primary T cells at the ACTB locus, a universally essential gene, and at the IL2RG locus, an immune cell specific essential gene, as indicated by the presence of GFP positive cells. This strategy is robust against variation between people, as shown by similar efficiencies across three different donors (Donors A, B, and C).

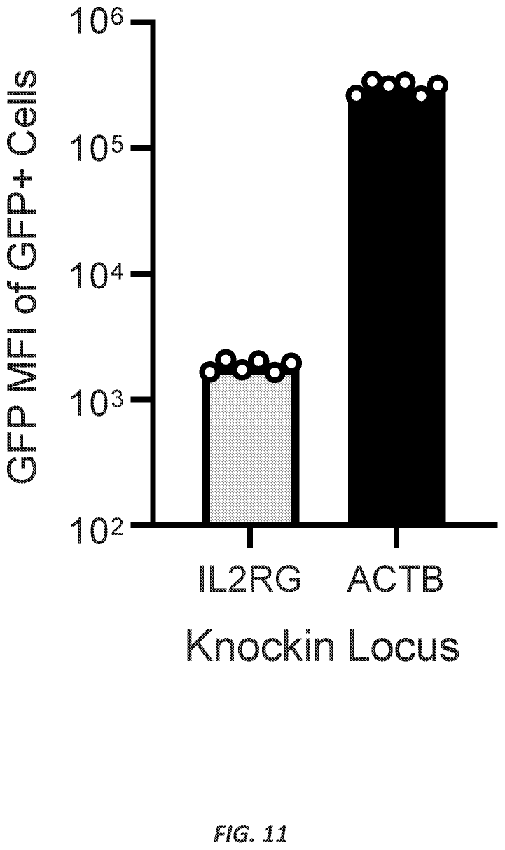

Additionally, by knocking in transgenes into genes with different endogenous promoter strengths, the transgene expression level can be modulated. This is demonstrated by knock-in of GFP upstream of two different genes, ACTB and IL2RG, in primary T cells. Because ACTB has a stronger endogenous promoter, knock-in of GFP upstream of ACTB leads to higher GFP expression, measured by flow cytometry, relative to knock-in upstream of IL2RG, as shown in FIG. 11.

### Example 8. Knock-In Method i Robust in Primary T Cells

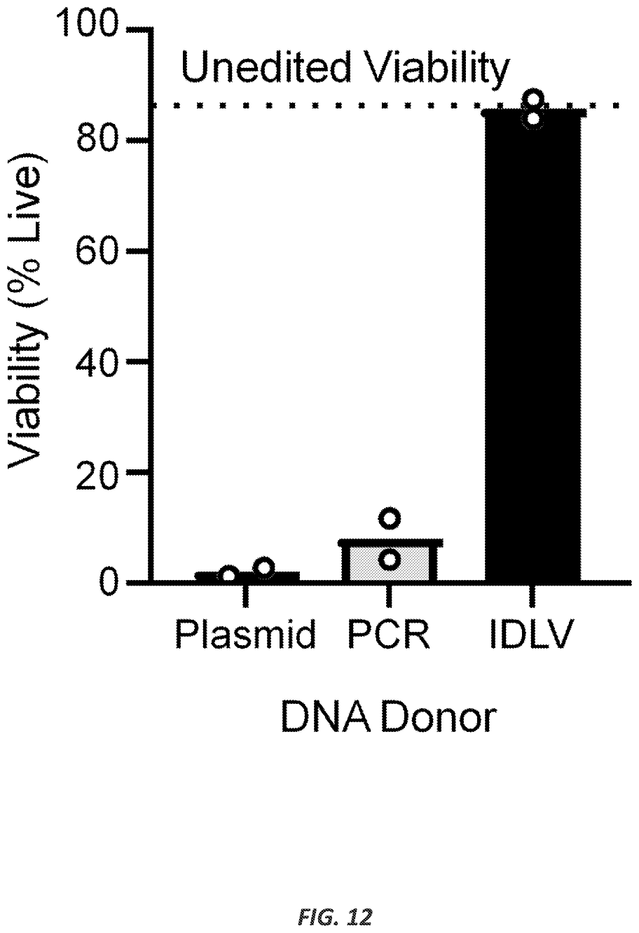

Previous knock-in methods in primary T cells suffer from toxicity and are prone to failing when small variations to the protocol are made. The knock-in methods described herein maintain the viability of untouched cells, as shown in FIG. 12. Fluorescent protein was knocked in to the ACTB locus as described in Example 1. To compare. 1,000,000 primary T cells were mixed with Cas9-RNP and either the same knock-in template in a plasmid backbone or as a PCR product. As previously described, the reaction was made by first adding template, then Cas9-RNP, then cells. Cells were then nucleofected and assessed for viability using a viability stain and flow cytometry 4 days later. PCR product and plasmid was utilized as it also could, in theory, allow for knock-ins of large payloads but suffered from extreme toxicity.

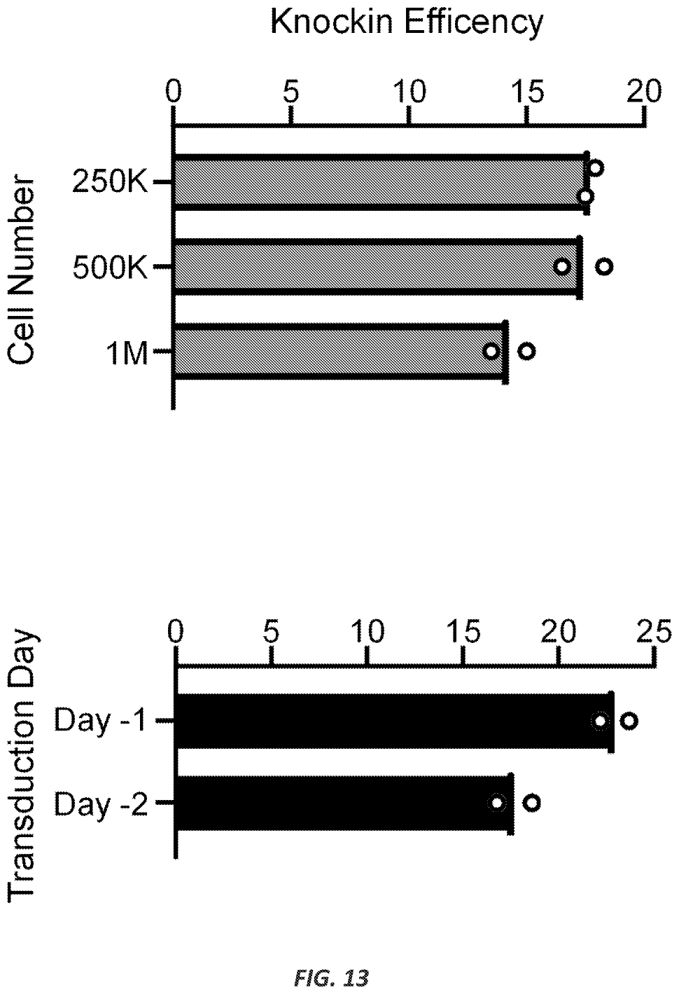

In addition, changes to the protocol did not result in large changes in efficiency, as shown in FIG. 13. Changing the number of primary T cells put into the nucleofection reaction and moving the day of IDLV transduction back 24 hours did not change the knockin efficiency, assayed by flow cytometry.

### Example 9. Knock-In Upstream of Essential Genes Promotes Stabilized Expression Relative to Traditional Methods

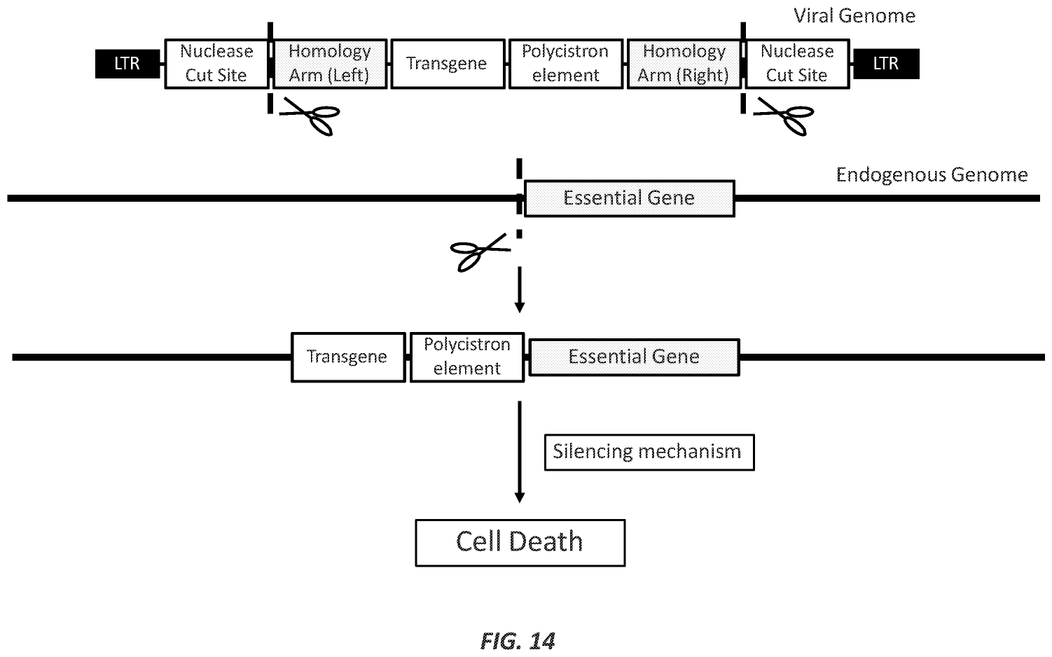

Transgene payloads introduced using traditional viral genetic engineering methods are prone to silencing. Knock-in of a transgene containing a polycistronic element, such as a P2A or IRES element, upstream of an essential gene (such as ACTB) stabilizes gene expression by creating selective pressure against transgene silencing. In other words, if the integrated transgene is silenced by the cell, expression of the essential gene will also be silenced, leading to cell death. A schematic representation of this knock-in strategy is shown in FIG. 14.

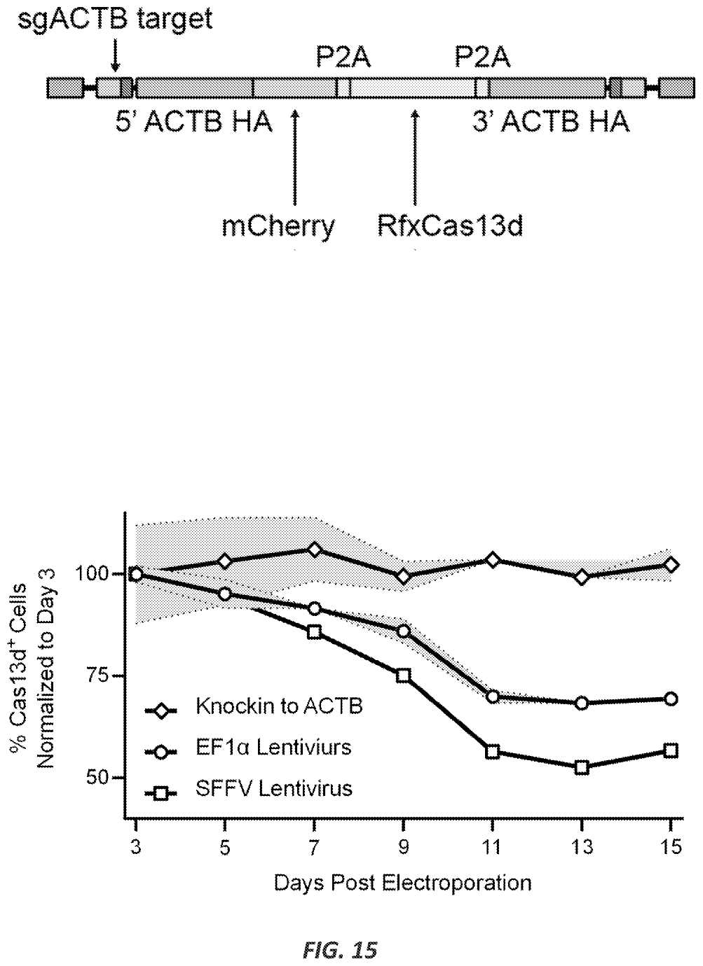

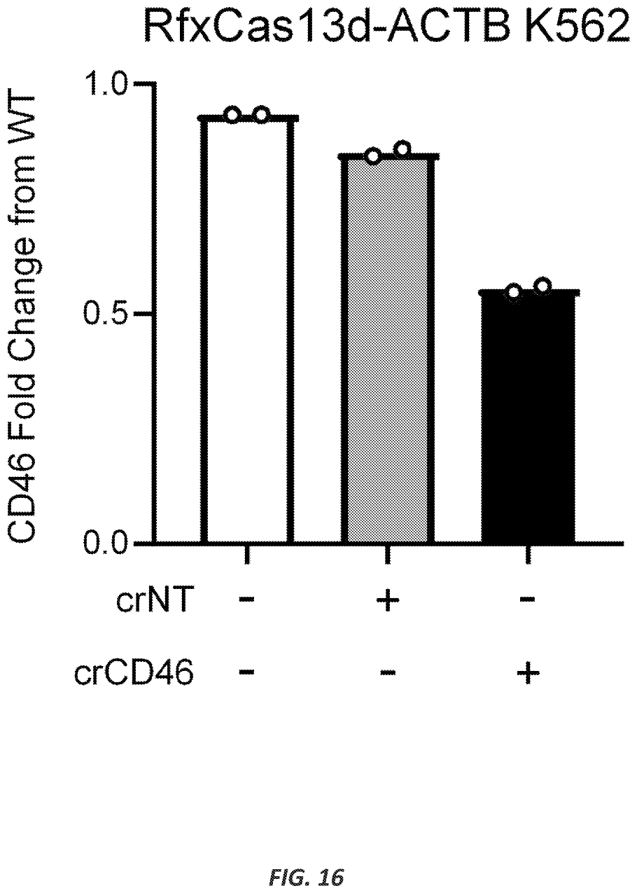

To compare the technique described in Example 5 to traditional viral transgene integration methods in primary T cells, a difficult to express payload, RfxCas13d, was knocked in upstream of ACTB under the control of the endogenous promoter or transduced using traditional lentiviral method under the control of synthetic promoters EF1α or SFFV. As shown in FIG. 15, traditional viral methods experience silencing of the transgene, while the knock-in method described herein remains stable over a 15 day period, as measured by flow cytometry. This transgene is functional, as shown in FIG. 16 where RfxCas13d was first knocked in to ACTB in K562 cells, then 3 days later a guide RNA for RfxCas13d targeting CD46 was introduced via lentivirus, which led to a reduction in CD46 expression as measured by surface stain and flow cytometry.

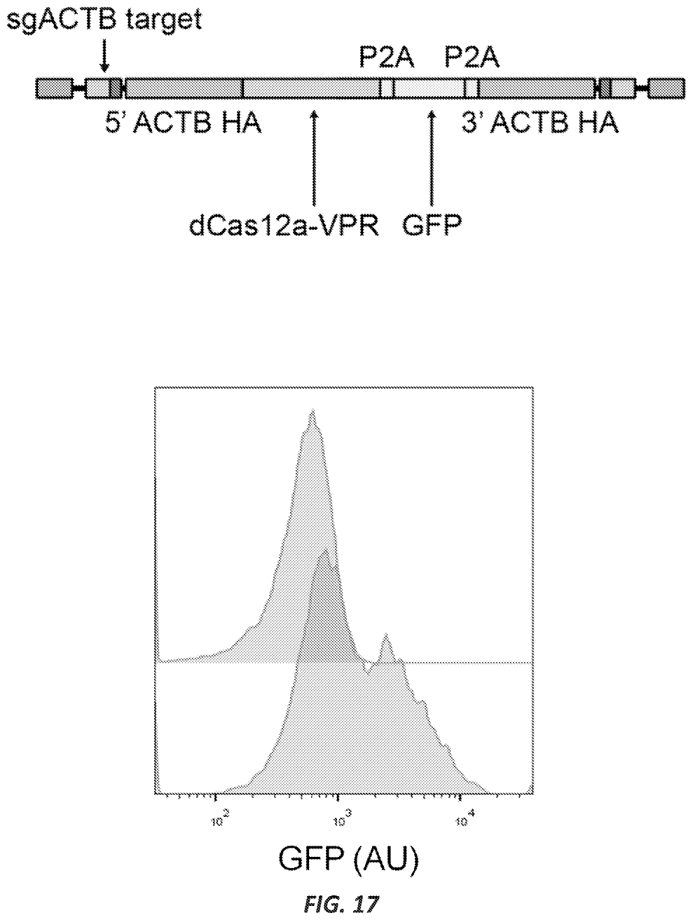

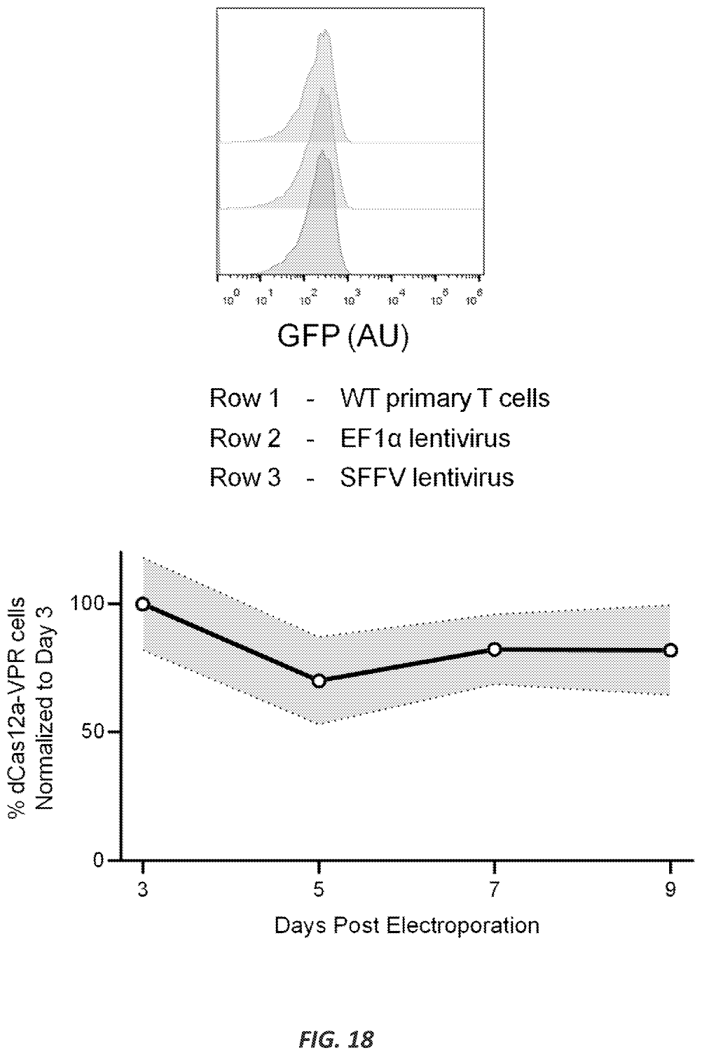

Another large and difficult to express gene, the CRISPR activation (CRISPRa) tool dCas12a-VPR, was knocked into the ACTB locus of K562 cells, leading to expression of the gene as shown in FIG. 17. As seen in FIG. 18, while methods using lentivirus to express the gene under the control of SFFV or EF1α led to silencing before the flow cytometry assay began, the method described herein led to stable expression of the gene over a 9 day period in primary T cells.

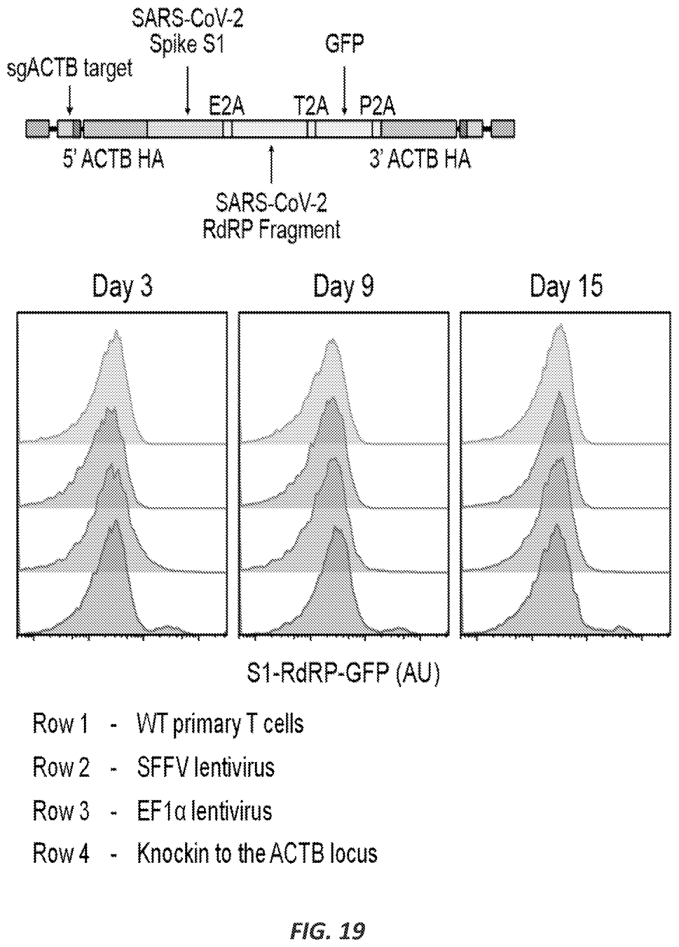

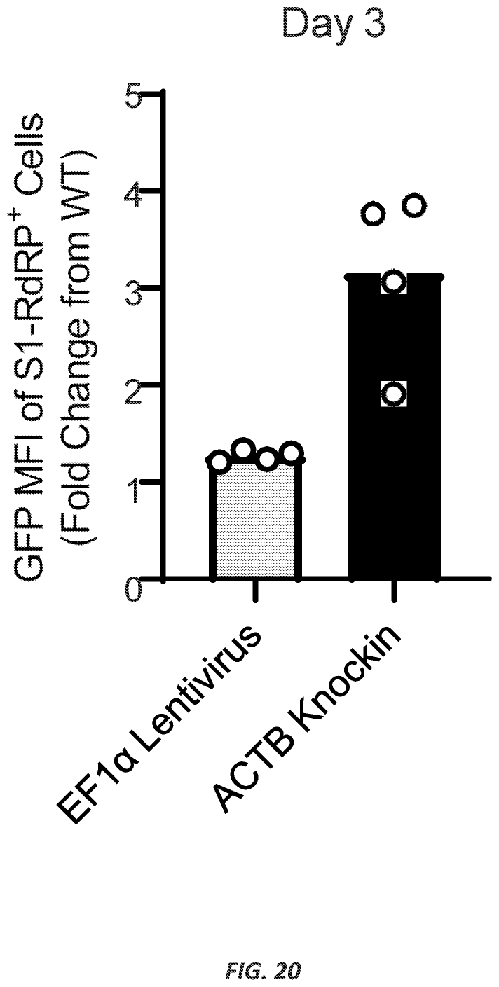

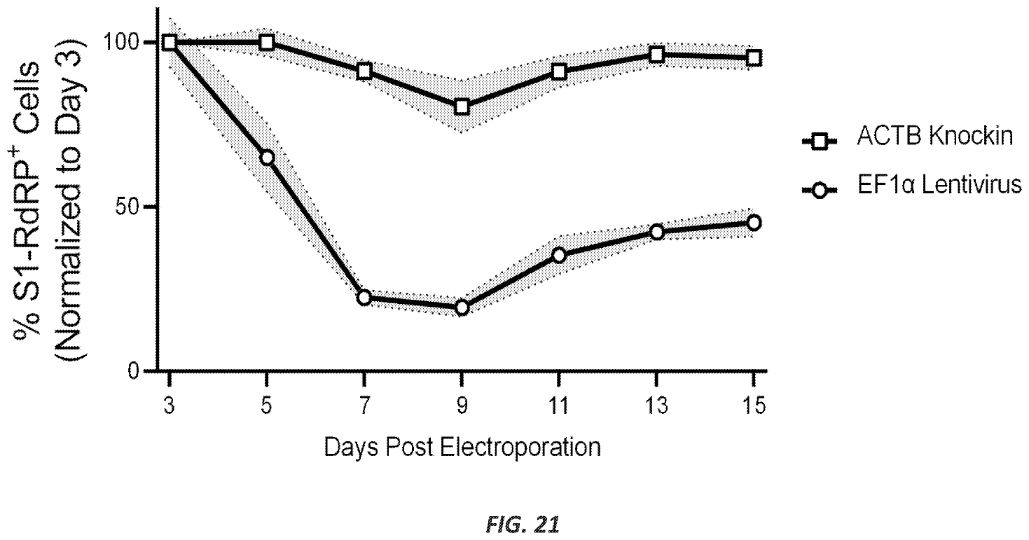

Additionally, a large and difficult to express payload, the toxic S1 domain from the SARS-CoV-2 Spike protein, the SARS-CoV-2 RNA dependent RNA polymerase, and GFP (5.7 kb total), was knocked in upstream of ACTB under control of the endogenous promoter (as described in Example 5) or transduced using traditional lentiviral method under the control of synthetic promoters EF1α or SFFV. As shown in FIG. 19 and FIG. 20, the efficiency of the described method (as measured by percentage of GFP positive cells using two different donor templates) was similar to the traditional approach using EF1α and more efficient than the traditional approach using SFFV when measured by flow cytometry 3 days post RNP nucleofection. However, when transgene expression was monitored for 15 days post electroporation as shown in FIG. 21, expression of the transgene knocked in to the ACTB locus under control of the endogenous promoter remains consistent, while expression of the transgene under control of the EF1α promoter decreases considerably over time, indicating transgene silencing.

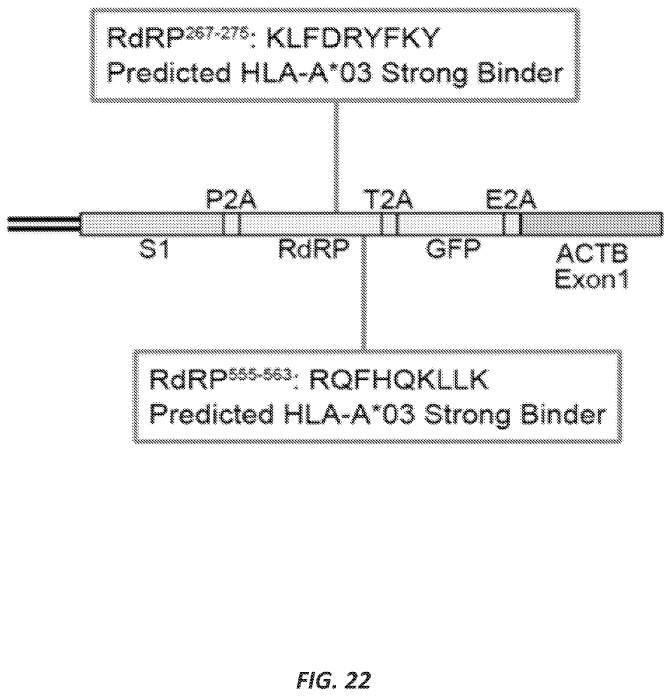

The toxic S1 domain from the SARS-CoV-2 Spike protein, the SARS-CoV-2 RNA dependent RNA polymerase, and GFP (5.7 kb total), was then knocked in upstream of ACTB in Jurkats. Cells were expanded and submitted to immunopeptidomics where peptide bound to MHCI was identified via mass spectrometry. As shown in FIG. 22, expression of the integrated transgene led to presentation of SARS-CoV-2 peptides which could be used for future basic research or for cell-based vaccines.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### Exemplary Embodiments

Exemplary embodiments provided in accordance with the presently disclosed subject matter include, but are not limited to, the claims and the following embodiments:
Embodiment 1: a donor template comprising: (a) a payload comprising a nucleotide sequence, (b) one or more homology arms comprising nucleotide sequences, wherein the nucleotide sequences are substantially identical to at least one locus in a genome, and (c) one or more cleavage sites comprising nucleotide sequences, wherein the nucleotide sequences can be bound or cleaved by a nuclease.
Embodiment 2: the donor template of embodiment 1, wherein the donor template is single-stranded.
Embodiment 3: the donor template of embodiment 1, wherein the donor template is double-stranded.
Embodiment 4: the donor template of embodiment 1, wherein the donor template is a plasmid or DNA fragment or vector.
Embodiment 5: the donor template of embodiment 4, wherein the donor template is a plasmid comprising elements necessary for replication, optionally comprising a promoter and a 3′ UTR.
Embodiment 6: the vector of embodiment 4, wherein the vector is a viral vector.
Embodiment 7: the vector of embodiment 6, wherein the vector is selected from the group consisting of retroviral, lentiviral adenoviral, adeno-associated viral, herpes simplex viral, Alphaviral, flaviviral, Rhabdoviral, Newcastle disease viral, Picornaviral, poxviral, Coxsackieviral, and measles viral vectors.
Embodiment 8: the vector of embodiment 6, wherein the vector is a modified viral vector selected from the group consisting of retroviral, lentiviral, adenoviral, adeno-associated viral, herpes simplex viral, Alphaviral, flaviviral, Rhabdoviral, Newcastle disease viral, Picornaviral, poxviral, Coxsackieviral, and measles viral vectors.
Embodiment 9: the vector of embodiment 7 or 8, wherein the vector is a retroviral vector.
Embodiment 10: the vector of embodiment 9, wherein the retroviral vector is a lentiviral vector.
Embodiment 11: the vector of any one of embodiments 6 to 10, further comprising genes necessary for replication, transcription, or reverse transcription of the viral vector.
Embodiment 12: the donor template or vector of any one of embodiments 1 to 11, wherein the genome is a mammalian genome.
Embodiment 13: the donor template or vector of embodiment 12, wherein the genome is a human genome.
Embodiment 14: the donor template or vector of any one of embodiments 1 to 13, wherein the payload comprises a nucleotide sequence of at least 4,400 nucleotides.
Embodiment 15: the donor template or vector of embodiment 14, wherein the payload comprises a nucleotide sequence of at least 4,700 nucleotides.
Embodiment 16: the donor template or vector of embodiment 14 or 15, wherein the payload comprises a nucleotide sequence of at least 6,000 nucleotides.
Embodiment 17: the donor template or vector of any one of embodiments 1 to 13, wherein the payload comprises a nucleotide sequence of up to 4,400 nucleotides.
Embodiment 18: the donor template or vector of any one of embodiments 1 to 13, wherein the payload comprises a nucleotide sequence of up to 4,700 nucleotides.
Embodiment 19: the donor template or vector of any one of embodiments 1 to 13, wherein the payload comprises a nucleotide sequence of up to 8,000 nucleotides.
Embodiment 20: the donor template or vector of any one of embodiments 1 to 13, wherein the payload comprises a nucleotide sequence of up to 8,500 nucleotides.
Embodiment 21: the donor template or vector of any one of embodiments 1 to 20, wherein the payload comprises a transgene.
Embodiment 22: the donor template or vector of embodiment 21, wherein the transgene does not comprise a promoter.
Embodiment 23: the donor template or vector of embodiment 22, wherein the transgene comprises a polycistronic expression element.
Embodiment 24: the donor template or vector of embodiment 23, wherein the polycistronic expression element is selected from the group consisting of: an IRES element, a P2A element, a T2A element, an E2A element, or an F2A element.
Embodiment 25: the donor template or vector of any one of embodiments 1 to 24, wherein the transgene comprises a translation enhancement element.
Embodiment 26: the donor template or vector of any one of embodiments 1 to 25, wherein the one or more homology arms independently comprise nucleotide sequences of up to 1,000 nucleotides.
Embodiment 27: the donor template or vector of any one of embodiments 1 to 26, wherein the one or more cleavage sites comprise nucleotide sequences that are substantially identical to a fragment of the at least one locus in the genome.
Embodiment 28: the donor template or vector of any one of embodiments 1 to 27, wherein the donor template or vector comprises at least two homology arms.
Embodiment 29: the donor template or vector of any one of embodiments 1 to 28, wherein the donor template or vector comprises at least two cleavage sites.
Embodiment 30: the donor template or vector of any one of embodiments 1 to 29, wherein the donor template or vector comprises at least two homology arms and at least two cleavage sites; and the payload, homology arms and cleavage sites are organized according to the following linear order: cleavage site, homology arm, payload, homology arm, cleavage site.
Embodiment 31: the donor template or vector of any one of embodiments 1 to 30, wherein the donor template or vector comprises two payloads.
Embodiment 32: the donor template or vector of embodiment 31, wherein the donor template or vector comprises at least four homology arms and at least four cleavage sites; and the two payloads, homology arms and cleavage sites are organized according to the following linear order: cleavage site, homology arm, payload 1, homology arm, cleavage site, cleavage site, homology arm, payload 2, homology arm, cleavage site.
Embodiment 33: a system for targeting integration of at least one payload into at least one genomic locus comprising: (a) the donor template or vector of any one of embodiments 1 to 32; and (b) a nuclease targeted to the at least one genomic locus.
Embodiment 34: the system of embodiment 33, wherein the genomic locus is in a mammalian genome.
Embodiment 35: the system of embodiment 34, wherein the genomic locus is in a human genome.
Embodiment 36: the system of any one of embodiments 33 to 35, wherein the nuclease is also targeted to the one or more cleavage sites in the donor template or vector.
Embodiment 37: the system of any one of embodiments 33 to 36, wherein the nuclease is selected from the group consisting of a CRISPR-associated protein (Cas), a meganuclease, a zinc finger nuclease (ZFN), a transcription activator-like effector nuclease (TALEN), an Argonaute protein, or a transposase.
Embodiment 38: the system of embodiment 37, wherein the nuclease is a Cas protein and wherein the system further comprises at least one guide nucleic acid to target the Cas protein to the at least one genomic locus.
Embodiment 39: the system of embodiment 38, wherein the Cas protein comprises at least one copy of a nuclear localization signal (NLS).
Embodiment 40: the system of embodiment 38 or 39, wherein the Cas protein is Cas9, Cas12, Cas14, a modified version of Cas9, a modified version of Cas12, or a modified version of Cas14.
Embodiment 41: the system of any one of embodiments 33 to 40, wherein the system comprises a vector and wherein the vector is a retroviral vector.
Embodiment 42: the system of embodiment 41, wherein the retroviral vector is a lentiviral vector.
Embodiment 43: a method of targeting integration of at least one payload into at least one genomic locus in a mammalian cell comprising: (a) introducing into said mammalian cell at least a first nuclease targeted to the at least one genomic locus; and (b) introducing into said mammalian cell a donor template or vector of any one of embodiments 1 to 32.
Embodiment 44: the method of embodiment 43, wherein the nuclease is also targeted to the one or more cleavage sites in the donor template or vector.
Embodiment 45: the method of embodiment 43 or 44, wherein the nuclease is selected from the group consisting of a CRISPR-associated protein (Cas), a meganuclease, a zinc finger nuclease (ZFN), a transcription activator-like effector nuclease (TALEN), an Argonaute protein, or a transposase.
Embodiment 46: the method of embodiment 45, wherein the nuclease is a Cas protein and wherein the method further comprises introducing into the mammalian cell at least one guide nucleic acid to target the nuclease to the at least one genomic locus.
Embodiment 47: the method of embodiment 46, wherein the Cas protein comprises at least one copy of a nuclear localization signal (NLS).
Embodiment 48: the method of embodiment 46 or 47, wherein the Cas protein is Cas9, Cas12, Cas14, a modified version of Cas9, a modified version of Cas12, or a modified version of Cas14.
Embodiment 49: the method of any one of embodiments 46 to 48, wherein introducing the nuclease comprises introducing into the mammalian cell a polypeptide or a nucleic acid encoding said polypeptide; and introducing the at least one guide nucleic acid comprises introducing into the mammalian cell the at least one guide nucleic acid or a nucleic acid encoding said at least one guide nucleic acid.
Embodiment 50: the method of any one of embodiments 43 to 49, wherein the method comprises introducing into the mammalian host cell a vector and wherein the vector is a retroviral vector.
Embodiment 51: the method of embodiment 50, wherein the retroviral vector is a lentiviral vector.
Embodiment 52: the method of embodiment 51, wherein a pseudovirus is used to introduce the lentiviral vector into the mammalian host cell.
Embodiment 53: the method of embodiment 52, wherein the pseudovirus is integration-deficient.
Embodiment 54: the method of embodiment 53, wherein the pseudovirus comprises a mutant integrase protein comprising a D64V substitution.
Embodiment 55: the method of any one of embodiments 43 to 54, wherein the at least one genomic locus comprises a gene with a promoter.
Embodiment 56: the method of embodiment 55. wherein the gene is highly expressed.
Embodiment 57: the method of embodiment 55 or 56, wherein the gene encodes a protein that is required for survival of the mammalian cell.
Embodiment 58: the method of any one of embodiments 55 to 57, wherein the gene is selected from the group consisting of beta-actin, cytochrome P450, ribosomal subunit S19, IL2 receptor gamma, and CD3 epsilon chain.
Embodiment 59: the method of any one of embodiments 55 to 58, wherein the gene is selected from the group consisting of beta-actin and IL2 receptor gamma.
Embodiment 60: the method of any one of embodiments 55 to 59, wherein the gene is selected from the group consisting of oncogenes, tumor suppressor genes, and lineage marker genes.
Embodiment 61: the method of any one of embodiments 55 to 60, wherein the payload comprises: (a) a transgene without a promoter; and (b) a polycistronic expression element, and wherein the promoter at the at least one genomic locus can drive expression of the transgene following integration of the payload at said at least one genomic locus.
Embodiment 62: the method of embodiment 61, wherein the promoter can drive expression of both the gene and the integrated transgene.
Embodiment 63: the method of embodiment 62, wherein the mammalian cell is selected against if it silences transgene expression.
Embodiment 64: the method of any one of embodiments 43 to 63, further comprising producing one or more single-stranded breaks at said at least one genomic locus.
Embodiment 65: the method of any one of embodiments 43 to 64, further comprising producing at least one double-stranded break at said at least one genomic locus.
Embodiment 66: the method of any one of embodiments 43 to 65, wherein the at least one genomic locus is modified by homologous recombination using said donor template or vector.
Embodiment 67: the method of any one of embodiments 43 to 66, wherein introducing the donor template or vector occurs at least 12 hours prior to introducing the nuclease.
Embodiment 68: the method of any one of embodiments 43 to 66, wherein introducing the donor template or vector occurs at the same time as introducing the nuclease.
Embodiment 69: a pseudovirus comprising the donor template or vector of any one of embodiments 1 to 32.
Embodiment 70: the pseudovirus of embodiment 69, wherein the pseudovirus is integration-deficient.
Embodiment 71: the pseudovirus of embodiment 70, wherein the pseudovirus comprises a mutant integrase protein comprising a D64V substitution.
Embodiment 72: the pseudovirus of any one of embodiments 69 to 71, wherein the donor template or vector is located between long terminal repeats (LTRs) in the lentiviral genome.
Embodiment 73: a system for targeting integration of at least one payload into at least one genomic locus comprising: (a) the pseudovirus of any one of embodiments 69 to 72; and (b) a nuclease targeted to the at least one genomic locus.
Embodiment 74: the system of embodiment 73, wherein the nuclease is also targeted to the one or more cleavage sites in the donor template or vector.
Embodiment 75: the system of embodiment 73 or 74, wherein the nuclease is selected from the group consisting of a CRISPR-associated protein (Cas), a meganuclease, a zinc finger nuclease (ZFN), a transcription activator-like effector nuclease (TALEN), an Argonaute protein, or a transposase.
Embodiment 76: the system of embodiment 75, wherein the nuclease is a Cas protein and wherein the system further comprises introducing into the mammalian cell at least one guide nucleic acid to target the nuclease to the at least one genomic locus.
Embodiment 77: the system of embodiment 76, wherein the Cas protein comprises at least one copy of a nuclear localization signal (NLS).
Embodiment 78: the system of embodiment 76 or 77, wherein the Cas protein is Cas9, Cas12, Cas14, a modified version of Cas9, a modified version of Cas12, or a modified version of Cas14.
Embodiment 79: the system of any one of embodiments 73 to 78, wherein the pseudovirus comprises a vector and wherein the vector is a retroviral vector.
Embodiment 80: the system of embodiment 79, wherein the retroviral vector is a lentiviral vector.
Embodiment 81: a modified mammalian cell comprising at least one payload integrated into its genome according to the method of any one of embodiments 43 to 68.
Embodiment 82: the modified mammalian cell of embodiment 81, wherein the mammalian cell is selected from the group consisting of primary human T cells, human dendritic cells, or mouse T cells.
Embodiment 83: the modified mammalian cell of embodiment 81, wherein the mammalian cell is a lymphocyte, a phagocytic cell, a granulocytic cell, or a dendritic cell.
Embodiment 84: the modified mammalian cell of embodiment 83, wherein the lymphocyte is a T cell, a B cell, or a natural killer (NK) cell.
Embodiment 85: the modified mammalian cell of embodiment 84, wherein the T cell is a CD4+ helper T cell or a CD8+ killer T cell.
Embodiment 86: the modified mammalian cell of embodiment 83, wherein the phagocytic cell is a monocyte or a macrophage.
Embodiment 87: the modified mammalian cell of embodiment 83, wherein the granulocytic cell is a neutrophil or a mast cell.
Embodiment 88: the modified mammalian cell of embodiment 81, wherein the mammalian cell is a stem cell or a progenitor cell.
Embodiment 89: the modified mammalian cell of embodiment 88, wherein the stem cell is an induced pluripotent stem cell (iPSC), an embryonic stem cell (ESC), an adult stem cell, or a mesenchymal stem cell (MSC).
Embodiment 90: the modified mammalian cell of embodiment 88, wherein the progenitor cell is a neural progenitor cell, a skeletal progenitor cell, a muscle progenitor cell, a fat progenitor cell, a heart progenitor cell, a chondrocyte, or a pancreatic progenitor cell.
Embodiment 91: the modified mammalian cell of any one of embodiments 81 to 90, wherein the at least one payload comprises a transgene expressing an antigen capable of inducing an immune response in a subject.
Embodiment 92: the modified mammalian cell of embodiment 91, wherein the antigen is a spike protein from a human coronavirus.
Embodiment 93: the modified mammalian cell of embodiment 92, wherein the spike protein is from human SARS-CoV-2.
Embodiment 94: the modified mammalian cell of embodiment 91, wherein the antigen is an RNA-dependent RNA polymerase (RdRP) protein from a human coronavirus.
Embodiment 95: the modified mammalian cell of embodiment 94, wherein the RdRP protein is from human SARS-CoV-2.
Embodiment 96: a vaccine comprising the modified mammalian cell of any one of embodiments 81 to 95.
Embodiment 97: the vaccine of embodiment 96, further comprising are excipient, an adjuvant, or a combination thereof.
Embodiment 98: a method of inducing an immune response in a subject comprising administering the modified mammalian cell of any one of embodiments 81 to 95 or the vaccine of embodiment 96 or 97 to the subject.
Embodiment 99: the method of embodiment 98, wherein administering the modified mammalian cell comprises infusing the modified mammalian cell into the subject.

SEQUENCE LISTING
Integration-deficient lentivirus
plasmid sequence
[SEQ ID NO: 1]
ttgattattgactagttattaatagtaatcaattacgggg
tcattagttcatagcccatatatggagttccgcgttacat
aacttacggtaaatggcccgcctggctgaccgcccaacga
cccccgcccattgacgtcaataatgacgtatgttcccata
gtaacgccaatagggactttccattgacgtcaatgggtgg
agtatttacggtaaactgcccacttggcagtacatcaagt
gtatcatatgccaagtacgccccctattgacgtcaatgac
ggtaaatggcccgcctggcattatgcccagtacatgacct
tatgggactttcctacttggcagtacatctacgtattagt
catcgctattaccatggtgatgcggttttggcagtacatc
aatgggcgtggatagcggtttgactcacggggatttccaa
gtctccaccccattgacgtcaatgggagtttgttttggca
ccaaaatcaacgggactttccaaaatgtcgtaacaactcc
gccccattgacgcaaatgggcggtaggcgtgtacggtggg
aggtctatataagcagagctcgtttagtgaaccgtcagat
cgcctggagacgccatccacgctgttttgacctccataga
agacaccgggaccgatccagcctccgcggccgggaacggt
gcattggaacgcggattccccgtgccaagagtgacgtaag
taccgcctatagagtctataggcccacccccttggcttct
tatgcgacggatcgatcccgtaataagcttcgaggtccgc
ggccggccgcgttgacgcgcacggcaagaggcgaggggcg
gcgactggtgagagatgggtgcgagagcgtcagtattaag
cgggggagaattagatcgatgggaaaaaattcggttaagg
ccagggggaaagaaaaaatataaattaaaacatatagtat
gggcaagcagggagctagaacgattcgcagttaatcctgg
cctgttagaaacatcagaaggctgtagacaaatactggga
cagctacaaccatcccttcagacaggatcagaagaactta
gatcattatataatacagtagcaaccctctattgtgtgca
tcaaaggatagagataaaagacaccaaggaagctttagac
aagatagaggaagagcaaaacaaaagtaagaaaaaagcac
agcaagcagcagctgacacaggacacagcaatcaggtcag
ccaaaattaccctatagtgcagaacatccaggggcaaatg
gtacatcaggccatatcacctagaactttaaatgcatggg
taaaagtagtagaagagaaggctttcagcccagaagtgat
acccatgttttcagcattatcagaaggagccaccccacaa
gatttaaacaccatgctaaacacagtggggggacatcaag
cagccatgcaaatgttaaaagagaccatcaatgaggaagc
tgcagaatgggatagagtgcatccagtgcatgcagggcct
attgcaccaggccagatgagagaaccaaggggaagtgaca
tagcaggaactactagtacccttcaggaacaaataggatg
gatgacacataatccacctatcccagtaggagaaatctat
aaaagatggataatcctgggattaaataaaatagtaagaa
tgtatagccctaccagcattctggacataagacaaggacc
aaaggaacccttagagactatgtagaccgattctataaaa
ctctaagagccgagcaagcttcacaagaggtaaaaaattg
gatgacagaaaccttgttggtccaaaatgcgaacccagat
tgtaagactattttaaaagcattgggaccaggagcgacac
tagaagaaatgatgacagcatgtcagggagtggggggacc
cggccataaagcaagagttttggctgaagcaatgagccaa
gtaacaaatccagctaccataatgatacagaaaggcaatt
ttaggaaccaaagaaagactgttaagtgtttcaattgtgg
caaagaagggcacatagccaaaaattgcagggcccctagg
aaaaagggctgttggaaatgtggaaaggaaggacaccaaa
tgaaagattgtactgagagacaggctaattttttagggaa
gatctggccttcccacaagggaaggccagggaattttctt
cagagcagaccagagccaacagccccaccagaagagagct
tcaggtttggggaagagacaacaactccctctcagaagca
ggagccgatagacaaggaactgtatcctttagcttccctc
agatcactctttggcagcgacccctcgtcacaataaagat
aggggggcaattaaaggaagctctattagatacaggagca
gatgatacagtattagaagaaatgaatttgccaggaagat
ggaaaccaaaaatgatagggggaattggaggttttatcaa
agtaagacagtatgatcagatactcatagaaatctgcgga
cataaagctataggtacagtattagtaggacctacacctg
tcaacataattggaagaaatctgttgactcagattggctg
cactttaaattttcccattagtcctattgagactgtacca
gtaaaattaaagccaggaatggatggcccaaaagttaaac
aatggccattgacagaagaaaaaataaaagcattagtaga
aatttgtacagaaatggaaaaggaaggaaaaatttcaaaa
attgggcctgaaaatccatacaatactccagtatttgcca
taaagaaaaaagacagtactaaatggagaaaattagtaga
tttcagagaacttaataagagaactcaagatttctgggaa
gttcaattaggaataccacatcctgcagggttaaaacaga
aaaaatcagtaacagtactggatgtgggcgatgcatattt
ttcagttcccttagataaagacttcaggaagtatactgca
tttaccatacctagtataaacaatgagacaccagggatta
gatatcagtacaatgtgcttccacagggatggaaaggatc
accagcaatattccagtgtagcatgacaaaaatcttagag
ccttttagaaaacaaaatccagacatagtcatctatcaat
acatggatgatttgtatgtaggatctgacttagaaatagg
gcagcatagaacaaaaatagaggaactgagacaacatctg
ttgaggtggggatttaccacaccagacaaaaaacatcaga
aagaacctccattcctttggatgggttatgaactccatcc
tgataaatggacagtacagcctatagtgctgccagaaaag
gacagctggactgtcaatgacatacagaaattagtgggaa
aattgaattgggcaagtcagatttatgcagggattaaagt
aaggcaattatgtaaacttcttaggggaaccaaagcacta
acagaagtagtaccactaacagaagaagcagagctagaac
tggcagaaaacagggagattctaaaagaaccggtacatgg
agtgtattatgacccatcaaaagacttaatagcagaaata
cagaagcaggggcaaggccaatggacatatcaaatttatc
aagagccatttaaaaatctgaaaacaggaaagtatgcaag
aatgaagggtgcccacactaatgatgtgaaacaattaaca
gaggcagtacaaaaaatagccacagaaagcatagtaatat
ggggaaagactcctaaatttaaattacccatacaaaagga
aacatgggaagcatggtggacagagtattggcaagccacc
tggattcctgagtgggagtttgtcaatacccctcccttag
tgaagttatggtaccagttagagaaagaacccataatagg
agcagaaactttctatgtagatggggcagccaatagggaa
actaaattaggaaaagcaggatatgtaactgacagaggaa
gacaaaaagttgtccccctaacggacacaacaaatcagaa
gactgagttacaagcaattcatctagctttgcaggattcg
ggattagaagtaaacatagtgacagactcacaatatgcat
tgggaatcattcaagcacaaccagataagagtgaatcaga
gttagtcagtcaaataatagagcagttaataaaaaaggaa
aaagtctacctggcatgggtaccagcacacaaaggaattg
gaggaaatgaacaagtagataaattggtcagtgctggaat
caggaaagtactatttttagatggaatagataaggcccaa
gaagaacatgagaaatatcacagtaattggagagcaatgg
ctagtgattttaacctaccacctgtagtagcaaaagaaat
agtagccagctgtgataaatgtcagctaaaaggggaagcc
atgcatggacaagtagactgtagcccaggaatatggcagc
tagtatgtacacatttagaaggaaaagttatcttggtagc
agttcatgtagccagtggatatatagaagcagaagtaatt
ccagcagagacagggcaagaaacagcatacttcctcttaa
aattagcaggaagatggccagtaaaaacagtacatacaga
caatggcagcaatttcaccagtactacagttaaggccgcc
tgttggtgggggggatcaagcaggaatttggcattcccta
caatccccaaagtcaaggagtaatagaatctatgaataaa
gaattaaagaaaattataggacaggtaagagatcaggctg
aacatcttaagacagcagtacaaatggcagtattcatcca
caattttaaaagaaaaggggggattggggggtacagtgca
ggggaaagaatagtagacataatagcaacagacatacaaa
ctaaagaattacaaaaacaaattacaaaaattcaaaattt
tcgggtttattacagggacagcagagatccagtttggaaa
ggaccagcaaagctcctctggaaaggtgaaggggcagtag
taatacaagataatagtgacataaaagtagtgccaagaag
aaaagcaaagatcatcagggattatggaaaacagatggca
ggtgatgattgtgtggcaagtagacaggatgaggattaac
acatggaattctgcaacaactgctgtttatccatttcaga
attgggtgtcgacatagcagaataggcgttactcgacaga
ggagagcaagaaatggagccagtagatcctagactagagc
cctggaagcatccaggaagtcagcctaaaactgcttgtac
caattgctattgtaaaaagtgttgctttcattgccaagtt
tgtttcatgacaaaagccttaggcatctcctatggcagga
agaagcggagacagcgacgaagagctcatcagaacagtca
gactcatcaagcttctctatcaaagcagtaagtagtacat
gtaatgcaacctataatagtagcaatagtagcattagtag
tagcaataataatagcaatagttgtgtggtccatagtaat
catagaatataggaaaatggccgctgatcttcagacctgg
aggaggagatatgagggacaattggagaagtgaattatat
aaatataaagtagtaaaaattgaaccattaggagtagcac
ccaccaaggcaaagagaagagtggtgcagagagaaaaaag
agcagtgggaataggagctttgttccttgggttcttggga
gcagcaggaagcactatgggcgcagcgtcaatgacgctga
cggtacaggccagacaattattgtctggtatagtgcagca
gcagaacaatttgctgagggctattgaggcgcaacagcat
ctgttgcaactcacagtctggggcatcaagcagctccagg
caagaatcctggctgtggaaagatacctaaaggatcaaca
gctcctggggatttggggttgctctggaaaactcatttgc
accactgctgtgccttggaatgctagttggagtaataaat
ctctggaacagatttggaatcacacgacctggatggagtg
ggacagagaaattaacaattacacaagcttaatacactcc
ttaattgaagaatcgcaaaaccagcaagaaaagaatgaac
aagaattattggaattagataaatgggcaagtttgtggaa
ttggtttaacataacaaattggctgtggtatataaaatta
ttcataatgatagtaggaggcttggtaggtttaagaatag
tttttgctgtactttctatagtgaatagagttaggcaggg
atattcaccattatcgtttcagacccacctcccaaccccg
aggggacccgacaggcccgaaggaatagaagaagaaggtg
gagagagagacagagacagatccattcgattagtgaacgg
atccttggcacttatctgggacgatctgcggagcctgtgc
ctcttcagctaccaccgcttgagagacttactcttgattg
taacgaggattgtggaacttctgggacgcaggggggggaa
gccctcaaatattggtggaatctcctacaatattggagtc
aggagctaaagaatagtgctgttagcttgctcaatgccac
agccatagcagtagctgaggggacagatagggttatagaa
gtagtacaaggagcttgtagagctattcgccacataccta
gaagaataagacagggcttggaaaggattttgctataagc
tcgaggccgccccggtgaccttcagaccttggcactggag
gtggcccggcagaagcgcggcatcgtggatcagtgctgca
ccagcatctgctctctctaccaactggagaactactgcaa
ctaggcccaccactaccctgtccacccctctgcaatgaat
aaaacctttgaaagagcactacaagttgtgtgtacatgcg
tgcatgtgcatatgtggtgcggggggaacatgagtggggc
tggctggagtggcgatgataagctgtcaaacatgagaatt
aattcttgaagacgaaagggcctcgtgatacgcctatttt
tataggttaatgtcatgataataatggtttcttagtctag
aattaattccgtgtattctatagtgtcacctaaatcgtat
gtgtatgatacataaggttatgtattaattgtagccgcgt
tctaacgacaatatgtacaagcctaattgtgtagcatctg
gcttactgaagcagaccctatcatctctctcgtaaactgc
cgtcagagtcggtttggttggacgaaccttctgagtttct
ggtaacgccgtcccgcacccggaaatggtcagcgaaccaa
tcagcagggtcatcgctagccagatcctctacgccggacg
catcgtggccggcatcaccggcgccacaggtgcggttgct
ggcgcctatatcgccgacatcaccgatggggaagatcggg
ctcgccacttcgggctcatgagcgcttgtttcggcgtggg
tatggtggcaggccccgtggccgggggactgttgggcgcc
atctccttgcatgcaccattccttgcggcggcggtgctca
acggcctcaacctactactgggctgcttcctaatgcagga
gtcgcataagggagagcgtcgaatggtgcactctcagtac
aatctgctctgatgccgcatagttaagccagccccgacac
ccgccaacacccgctgacgcgccctgacgggcttgtctgc
tcccggcatccgcttacagacaagctgtgaccgtctccgg
gagctgcatgtgtcagaggttttcaccgtcatcaccgaaa
cgcgcgagacgaaagggcctcgtgatacgcctatttttat
aggttaatgtcatgataataatggtttcttagacgtcagg
tggcacttttcggggaaatgtgcgcggaacccctatttgt
ttatttttctaaatacattcaaatatgtatccgctcatga
gacaataaccctgataaatgcttcaataatattgaaaaag
gaagagtatgagtattcaacatttccgtgtcgcccttatt
cccttttttgcggcattttgccttcctgtttttgctcacc
cagaaacgctggtgaaagtaaaagatgctgaagatcagtt
gggtgcacgagtgggttacatcgaactggatctcaacagc
ggtaagatccttgagagttttcgccccgaagaacgttttc
caatgatgagcacttttaaagttctgctatgtggcgcggt
attatcccgtattgacgccgggcaagagcaactcggtcgc
cgcatacactattctcagaatgacttggttgagtactcac
cagtcacagaaaagcatcttacggatggcatgacagtaag
agaattatgcagtgctgccataaccatgagtgataacact
gcggccaacttacttctgacaacgatcggaggaccgaagg
agctaaccgcttttttgcacaacatgggggatcatgtaac
tcgccttgatcgttgggaaccggagctgaatgaagccata
ccaaacgacgagcgtgacaccacgatgcctgtagcaatgg
caacaacgttgcgcaaactattaactggcgaactacttac
tctagcttcccggcaacaattaatagactggatggaggcg
gataaagttgcaggaccacttctgcgctcggcccttccgg
ctggctggtttattgctgataaatctggagccggtgagcg
tgggtctcgcggtatcattgcagcactggggccagatggt
aagccctcccgtatcgtagttatctacacgacggggagtc
aggcaactatggatgaacgaaatagacagatcgctgagat
aggtgcctcactgattaagcattggtaactgtcagaccaa
gtttactcatatatactttagattgatttaaaacttcatt
tttaatttaaaaggatctaggtgaagatcctttttgataa
tctcatgaccaaaatcccttaacgtgagttttcgttccac
tgagcgtcagaccccgtagaaaagatcaaaggatcttctt
gagatcctttttttctgcgcgtaatctgctgcttgcaaac
aaaaaaaccaccgctaccagcggtggtttgtttgccggat
caagagctaccaactctttttccgaaggtaactggcttca
gcagagcgcagataccaaatactgttcttctagtgtagcc
gtagttaggccaccacttcaagaactctgtagcaccgcct
acatacctcgctctgctaatcctgttaccagtggctgctg
ccagtggcgataagtcgtgtcttaccgggttggactcaag
acgatagttaccggataaggcgcagcggtcgggctgaacg
gggggttcgtgcacacagcccagcttggagcgaacgacct
acaccgaactgagatacctacagcgtgagctatgagaaag
cgccacgcttcccgaagggagaaaggcggacaggtatccg
gtaagcggcagggtcggaacaggagagcgcacgagggagc
ttccagggggaaacgcctggtatctttatagtcctgtcgg
gtttcgccacctctgacttgagcgtcgatttttgtgatgc
tcgtcaggggggcggagcctatggaaaaacgccagcaacg
cggcctttttacggttcctggccttttgctggccttttgc
tcacatgttctttcctgcgttatcccctgattctgtggat
aaccgtattaccgcctttgagtgagctgataccgctcgcc
gcagccgaacgaccgagcgcagcgagtcagtgagcgagga
agcggaagagcgcccaatacgcaaaccgcctctccccgcg
cgttggccgattcattaatgcagctgtggaatgtgtgtca
gttagggtgtggaaagtccccaggctccccagcaggcaga
agtatgcaaagcatgcatctcaattagtcagcaaccaggt
gtggaaagtccccaggctccccagcaggcagaagtatgca
aagcatgcatctcaattagtcagcaaccatagtcccgccc
ctaactccgcccatcccgcccctaactccgcccagttccg
cccattctccgccccatggctgactaattttttttattta
tgcagaggccgaggccgcctcggcctctgagctattccag
aagtagtgaggaggcttttttggaggcctaggcttttgca
aaaagcttggacacaagacaggcttgcgagatatgtttga
gaataccactttatcccgcgtcagggagaggcagtgcgta
aaaagacgcggactcatgtgaaatactggtttttagtgcg
ccagatctctataatctcgcgcaacctattttcccctcga
acactttttaagccgtagataaacaggctgggacacttca
catgagcgaaaaatacatcgtcacctgggacatgttgcag
atccatgcacgtaaactcgcaagccgactgatgccttctg
aacaatggaaaggcattattgccgtaagccgtggcggtct
gtaccgggtgcgttactggcgcgtgaactgggtattcgtc
atgtcgataccgtttgtatttccagctacgatcacgacaa
ccagcgcgagcttaaagtgctgaaacgcgcagaaggcgat
ggcgaaggcttcatcgttattgatgacctggtggataccg
gtggtactgcggttgcgattcgtgaaatgtatccaaaagc
gcactttgtcaccatcttcgcaaaaccggctggtcgtccg
ctggttgatgactatgttgttgatatcccgcaagatacct
ggattgaacagccgtgggatatgggcgtcgtattcgtccc
gccaatctccggtcgctaatcttttcaacgcctggcactg
ccgggcgttgttctttttaacttcaggcgggttacaatag
tttccagtaagtattctggaggctgcatccatgacacagg
caaacctgagcgaaaccctgttcaaaccccgctttaaaca
tcctgaaacctcgacgctagtccgccgctttaatcacggc
gcacaaccgcctgtgcagtcggcccttgatggtaaaacca
tccctcactggtatcgcatgattaaccgtctgatgtggat
ctggcgcggcattgacccacgcgaaatcctcgacgtccag
gcacgtattgtgatgagcgatgccgaacgtaccgacgatg
atttatacgatacggtgattggctaccgtggcggcaactg
gatttatgagtgggccccggatctttgtgaaggaacctta
cttctgtggtgtgacataattggacaaactacctacagag
atttaaagctctaaggtaaatataaaatttttaacccgga
tctttgtgaaggaaccttacttctgtggtgtgacataatt
ggacaaactacctacagagatttaaagctctaaggtaaat
ataaaatttttaagtgtataatgtgttaaactactgattc
taattgtttgtgtattttagattccaacctatggaactga
tgaatgggagcagtggtggaatgcctttaatgaggaaaac
ctgttttgctcagaagaaatgccatctagtgatgatgagg
ctactgctgactctcaacattctactcctccaaaaaagaa
gagaaaggtagaagaccccaaggactttccttcagaattg
ctaagttttttgagtcatgctgtgtttagtaatagaactc
ttgcttgctttgctatttacaccacaaaggaaaaagctgc
actgctatacaagaaaattatggaaaaatattctgtaacc
tttataagtaggcataacagttataatcataacatactgt
tttttcttactccacacaggcatagagtgtctgctattaa
taactatgctcaaaaattgtgtacctttagctttttaatt
tgtaaaggggttaataaggaatatttgatgtatagtgcct
tgactagagatcataatcagccataccacatttgtagagg
ttttacttgctttaaaaaacctcccacacctccccctgaa
cctgaaacataaaatgaatgcaattgttgttgttgggctg
caggaattaattcgagctcgcccgaca
SV40 large T-antigen NLS
[SEQ ID NO: 2]
PKKKRKV
Nucleoplasmin bipartite NLS
[SEQ ID NO: 3]
KRPAATKKAGQAKKKK
c-myc NLS 1
[SEQ ID NO: 4]
PAAKRVKLD
c-myc NLS 2
[SEQ ID NO: 5]
RQRRNELKRSP
hRNPA1 M9 NLS
[SEQ ID NO: 6]
NQSSNFGPMKGGNFGGRSSGPYGGGGQYFAKPRNQGGY
IBB domain from importin-alpha
[SEQ ID NO: 7]
RMRIZFKNKGKDTAELRRRRVEVSVELRK
AKKDEQILKRRNV
Myoma T protein NLS 1
[SEQ ID NO: 8]
VSRKRPRP
Myoma T protein NLS 2
[SEQ ID NO: 9]
PPKKARED
human p53 NLS
[SEQ ID NO: 10]
PQPKKKPL
Mouse c-abl IV NLS
[SEQ ID NO: 11]
SALIKKKKKMAP
Influenza virus NS1 NLS 1
[SEQ ID NO: 12]
DRLRR
Influenza virus NS1 NLS 2
[SEQ ID NO: 13]
PKQKKRK
Hepatitis virus delta antigen NLS
[SEQ ID NO: 14]
RKLKKKIKKL
Mouse Mx1 protein NLS
[SEQ ID NO: 15]
REKKKFLKRR
Human poly(ADP-ribose) polymerase NLS
[SEQ ID NO: 16]
KRKGDEVDGVDEVAKKKSKK
Human steroid hormone receptors
glucocorticoid NLS
[SEQ ID NO: 17]
RKCLQAGMNLEARKTKK

What is claimed is:

1. A donor template comprising:
(a) a payload comprising a nucleotide sequence,
(b) one or more homology arms comprising nucleotide sequences, wherein the nucleotide sequences are substantially identical to at least one locus in a genome, and
(c) one or more cleavage sites comprising nucleotide sequences, wherein the nucleotide sequences can be bound or cleaved by a nuclease.

2. The donor template of claim 1, wherein the donor template is single-stranded.

3. The donor template of claim 1, wherein the donor template is double-stranded.

4. The donor template of claim 1, wherein the donor template is a plasmid or DNA fragment or vector.

5. The donor template of claim 4, wherein the donor template is a plasmid comprising elements necessary for replication, optionally comprising a promoter and a 3′ UTR.

6. The vector of claim 4, wherein the vector is a viral vector.

7. The vector of claim 6, wherein the vector is selected from the group consisting of retroviral, lentiviral, adenoviral, adeno-associated viral, herpes simplex viral, Alphaviral, flaviviral, Rhabdoviral, Newcastle disease viral, Picornaviral, poxviral, Coxsackieviral, and measles viral vectors.

8. The vector of claim 6, wherein the vector is a modified viral vector selected from the group consisting of retroviral, lentiviral, adenoviral, adeno-associated viral, herpes simplex viral, Alphaviral, Rhabdoviral, Newcastle disease viral, Picornaviral, poxviral, Coxsackieviral, and measles viral vectors.

9. The vector of claim 6, wherein the vector is a retroviral vector.

10. The vector of claim 9, wherein the retroviral vector is a lentiviral vector.

11. The vector of claim 6, further comprising genes necessary for replication, transcription, or reverse transcription of the viral vector.

12. The donor template of claim 1, wherein the genome is a mammalian genome.

13. The donor template of claim 12, herein the genome is a human genome.

14. The donor template of claim 1, wherein the payload comprises a nucleotide sequence of at least 4,400 nucleotides.

15. The donor template of claim 14, wherein the payload comprises a nucleotide sequence of at least 4,700 nucleotides.

16. The donor template claim 14, wherein the payload comprises a nucleotide sequence of at least 6,000 nucleotides.

17. The donor template of claim 1, wherein the payload comprises a nucleotide sequence of up to 4,400 nucleotides.

18. The donor template of claim 1, wherein the payload comprises a nucleotide sequence of up to 4,700 nucleotides.

19. The donor template of claim 1, wherein the payload comprises a nucleotide sequence of up to 8,000 nucleotides.

20. The donor template of claim 1 wherein the payload comprises a nucleotide sequence of up to 8,500 nucleotides.

21. The donor template of claim 1, wherein the payload comprises a transgene.

22. The donor template of claim 21, wherein the transgene does not comprise a promoter.

23. The donor template of claim 22, wherein the transgene comprises a polycistronic expression element.

24. The donor template of claim 23 wherein the polycistronic expression element is selected from the group consisting of: an IRES element, a P2A element, a T2A element, an E2A element, or an F2A element.

25. The donor template of claim 1, wherein the transgene comprises a translation enhancement element.

26. The donor template of claim 1, wherein the one or more homology arms independently comprise nucleotide sequences of up to 1,000 nucleotides.

27. The donor template of claim 1, wherein the one or more cleavage sites comprise nucleotide sequences that are substantially identical to a fragment of the at least one locus in the genome.

28. The donor template of claim 1, wherein the donor template comprises at least two homology arms.

29. The donor template of claim wherein the donor template comprises at least two cleavage sites.

30. The donor template of claim 1, wherein
the donor template comprises at least two homology arms and at least two cleavage sites; and
the payload, homology arms and cleavage sites are organized according to the following linear order: cleavage site, homology arm, payload, homology arm, cleavage site.

31. The donor template of claim 1, wherein the donor template comprises two payloads.

32. The donor template of claim 31, wherein
the donor template comprises at least four homology arms and at least four cleavage sites; and
the two payloads, homology arms and cleavage sites are organized according to the following linear order: cleavage site, homology arm, payload 1, homology arm, cleavage site, cleavage site, homology arm, payload 2, homology arm, cleavage site.

33. A system for targeting integration of at least one payload into at least one genomic locus comprising:
(a) the donor template of claim 1; and
(b) a nuclease targeted to the at least one genomic locus.

34. The system of claim 33, wherein the genomic locus is in a mammalian genome.

35. The system of claim 34, wherein the genomic locus is in a human genome.

36. The system of claim 33, wherein the nuclease is also targeted to the one or more cleavage sites in the donor template.

37. The system of claim 33, wherein the nuclease is selected from the group consisting of a CRISPR-associated protein (Cas), a meganuclease, a zinc finger nuclease (ZFN), a transcription activator-like effector nuclease (TALEN), an Argonaute protein, or a transposase.

38. The system of claim 37, wherein the nuclease is a Cas protein and wherein the system further comprises at least one guide nucleic acid to target the Cas protein to the at least one genomic locus.

39. The system of claim 38, wherein the Cas protein comprises at least one copy of a nuclear localization signal (NLS).

40. The system of claim 38, wherein the Cas protein is Cas9, Cas12, Cas14, a modified version of Cas9, a modified version of Cas12, or a modified version of Cas14.

41. A system for targeting integration of at least one payload into at least one genomic locus comprising:
(a) the vector of claim 4; and
(b) a nuclease targeted to the at least one genomic locus.

42. The system of claim 41, wherein the vector is a retroviral vector.

43. The system of claim 42, wherein the retroviral vector is a lentiviral vector.

44. A method of targeting integration of at least one payload into at least one genomic locus in a mammalian cell comprising:
(a) introducing into said mammalian cell at least a first nuclease targeted to the at least one genomic locus; and
(b) introducing into said mammalian cell the donor template of claim 1.

45. The method of claim 44, wherein the nuclease is also targeted to the one or more cleavage sites in the donor template.

46. The method of claim 44, wherein the nuclease is selected from the group consisting of a CRISPR-associated protein (Cas), a meganuclease, a zinc finger nuclease (ZFN), a transcription activator-like effector nuclease (TALEN), an Argonaute protein, or a transposase.

47. The method of claim 46, wherein the nuclease is a Cas protein and wherein the method further comprises introducing into the mammalian cell at least one guide nucleic acid to target the nuclease to the at least one genomic locus.

48. The method of claim 47, wherein the Cas protein comprises at least one copy of a nuclear localization signal (NLS).

49. The method of claim 47, wherein the Cas protein is Cas9, Cas12, Cas14, a modified version of Cas9, a modified version of Cas12, or a modified version of Cas14.

50. The method of claim 47, wherein
introducing the nuclease comprises introducing into the mammalian cell a polypeptide or a nucleic acid encoding said polypeptide; and
introducing the at least one guide nucleic acid comprises introducing into the mammalian cell the at least one guide nucleic acid or a nucleic acid encoding said at least one guide nucleic acid.

51. A method of targeting integration of at least one payload into at least one genomic locus in a mammalian cell comprising:
(a) introducing into said mammalian cell at least a first nuclease targeted to the at least one genomic locus; and
(b) introducing into said mammalian cell the vector of claim 4.

52. The method of claim 51, wherein the vector is a retroviral vector.

53. The method of claim 52, wherein the retroviral vector is a lentiviral vector.

54. The method of claim 53, wherein a pseudovirus is used to introduce the lentiviral vector into the mammalian host cell.

55. The method of claim 54, wherein the pseudovirus is integration-deficient.

56. The method of claim 55, wherein the pseudovirus comprises a mutant integrase protein comprising a D64V substitution.

57. The method of claim 44, wherein the at least one genomic locus comprises a gene with a promoter.

58. The method of claim 57, wherein the gene is highly expressed.

59. The method of claim 57, wherein the gene encodes a protein that is required for survival of the mammalian cell.

60. The method of claim 57, wherein the gene is selected from the group consisting of beta-actin, cytochrome P450, ribosomal subunit S19, IL2 receptor gamma, and CD3 epsilon chain.

61. The method of claim 57, wherein the gene is selected from the group consisting of beta-actin and IL2 receptor gamma.

62. The method of claim 57, wherein the gene is selected from the group consisting of oncogenes, tumor suppressor genes, and lineage marker genes.

63. The method of claim 57, wherein the payload comprises:
(a) a transgene without a promoter; and
(b) a polycistronic expression element,
and wherein the promoter at the at least one genomic locus can drive expression of the transgene following integration of the payload at said at least one genomic locus.

64. The method of claim 63, wherein the promoter can drive expression of both the gene and the integrated transgene.

65. The method of claim 64, wherein the mammalian cell is selected against if it silences transgene expression.

66. The method of claim 44, further comprising producing one or more single-stranded breaks at said at least one genomic locus.

67. The method of claim 44, further comprising producing at least one double-stranded break at said at least one genomic locus.

68. The method of claim 44, wherein the at least one genomic locus is modified by homologous recombination using said donor template.

69. The method of claim 44, wherein introducing the donor template occurs at least 12 hours prior to introducing the nuclease.

70. The method of claim 44, wherein introducing the donor template occurs at the same time as introducing the nuclease.

71. A pseudovirus comprising the donor template of claim 1.

72. The pseudovirus of claim 71, wherein the pseudovirus is integration-deficient.

73. The pseudovirus of claim 72, wherein the pseudovirus comprises a mutant integrase protein comprising a D64V substitution.

74. The pseudovirus of claim 71, wherein the donor template is located between long terminal repeats (LTRs) in the lentiviral genome.

75. A system for targeting integration of at least one payload into at least one genomic locus comprising:
(a) the pseudovirus of claim 71; and
(b) a nuclease targeted to the at least one genomic locus.

76. The system of claim 75, wherein the nuclease is also targeted to the one or more cleavage sites in the donor template.

77. The system of claim 75, wherein the nuclease is selected from the group consisting of a CRISPR-associated protein (Cas), a meganuclease, a zinc finger nuclease (ZFN), a transcription activator-like effector nuclease (TALEN), an Argonauts protein, or a transposase.

78. The system of claim 77, wherein the nuclease is a Cas protein and wherein the system further comprises introducing into the mammalian cell at least one guide nucleic acid to target the nuclease to the at least one genomic locus.

79. The system of claim 78, wherein the Cas protein comprises at least one copy of a nuclear localization signal (NLS).

80. The system of claim 78, wherein the Cas protein is Cas9, Cas12, Cas14, a modified version of Cas9, a modified version of Cas12, or a modified version of Cas14.

81. A system for targeting integration of at least one payload into at least one genomic locus comprising:
(a) a pseudovirus comprising the vector of claim 4; and
(b) a nuclease targeted to the at least one genomic locus.

82. The system of claim 81, wherein the vector is a retroviral vector.

83. The system of claim 82, wherein the retroviral vector is a lentiviral vector.

84. A modified mammalian cell comprising at least one payload integrated into its genome according to the method of claim 44.

85. The modified mammalian cell of claim 84, wherein the mammalian cell is selected from the group consisting of primary human T cells, human dendritic cells, or mouse T cells.

86. The modified mammalian cell of claim 84, wherein the mammalian cell is a lymphocyte, a phagocytic cell, a granulocytic cell, or a dendritic cell.

87. The modified mammalian cell of claim 86, wherein the lymphocyte is a T cell, a B cell, or a natural killer (NK) cell.

88. The modified mammalian cell of claim 87, wherein the T cell is a CD4+ helper T cell or a CD8+ killer T cell.

89. The modified mammalian cell of claim 86, wherein the phagocytic cell is a monocyte or a macrophage.

90. The modified mammalian cell of claim 86, wherein the granulocytic cell is a neutrophil or a mast cell.

91. The modified mammalian cell of claim 84, wherein the mammalian cell is a stem cell or a progenitor cell.

92. The modified mammalian cell of claim 91, wherein the stem cell is an induced pluripotent stem cell (iPSC), an embryonic stem cell (ESC), an adult stem cell, or a mesenchymal stem cell (MSC).

93. The modified mammalian cell of claim 91, wherein the progenitor cell is a neural progenitor cell, a skeletal progenitor cell, a muscle progenitor cell, a fat progenitor cell, a heart progenitor cell, a chondrocyte, or a pancreatic progenitor cell.

94. The modified mammalian cell of claim 84, wherein the at least one payload comprises a transgene expressing an antigen capable of inducing an immune response in a subject.

95. The modified mammalian cell of claim 94, wherein the antigen is a spike protein from a human coronavirus.

96. The modified mammalian cell of claim 95, wherein the spike protein is from human SARS-CoV-2.

97. The modified mammalian cell of claim 94, wherein the antigen is an RNA-dependent RNA polymerase (RdRP) protein from a human coronavirus.

98. The modified mammalian cell of claim 97, wherein the RdRP protein is from human SARS-CoV-2.

99. A vaccine comprising the modified mammalian cell of claim 84.

100. The vaccine of claim 99, further comprising an excipient, an adjuvant, or a combination thereof.

101. A method of inducing an immune response in a subject, the method comprising administering the modified mammalian cell of claim 84 to the subject.

102. The method of claim 101, wherein administering the modified mammalian cell comprises infusing the modified mammalian cell into the subject.


SEQUENCE LISTING (19 sequences)

SEQ ID NO: 1 - DNA, 12150 bp, Artificial Sequence
  Description of Artificial Sequence Synthetic polynucleotide
ttgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcgatcccgtaataagcttcgaggtccgcggccggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagtatgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagctcgaggccgccccggtgaccttcagaccttggcactggaggtggcccggcagaagcgcggcatcgtggatcagtgctgcaccagcatctgctctctctaccaactggagaactactgcaactaggcccaccactaccctgtccacccctctgcaatgaataaaacctttgaaagagcactacaagttgtgtgtacatgcgtgcatgtgcatatgtggtgcggggggaacatgagtggggctggctggagtggcgatgataagctgtcaaacatgagaattaattcttgaagacgaaagggcctcgtgatacgcctatttttataggttaatgtcatgataataatggtttcttagtctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaacccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttgggctgcaggaattaattcgagctcgcccgaca

SEQ ID NO: 2 - protein, 7 aa, Artificial Sequence
  Description of Artificial Sequence Synthetic peptide
PKKKRKV

SEQ ID NO: 3 - protein, 16 aa, Artificial Sequence
  Description of Artificial Sequence Synthetic peptide
KRPAATKKAGQAKKKK

SEQ ID NO: 4 - protein, 9 aa, Artificial Sequence
  Description of Artificial Sequence Synthetic peptide
PAAKRVKLD

SEQ ID NO: 5 - protein, 11 aa, Artificial Sequence
  Description of Artificial Sequence Synthetic peptide
RQRRNELKRSP

SEQ ID NO: 6 - protein, 38 aa, Artificial Sequence
  Description of Artificial Sequence Synthetic polypeptide
NQSSNFGPMKGGNFGGRSSGPYGGGGQYFAKPRNQGGY

SEQ ID NO: 7 - protein, 42 aa, Artificial Sequence
  Description of Artificial Sequence Synthetic polypeptide
RMRIZFKNKGKDTAELRRRRVEVSVELRKAKKDEQILKRRNV

SEQ ID NO: 8 - protein, 8 aa, Artificial Sequence
  Description of Artificial Sequence Synthetic peptide
VSRKRPRP

SEQ ID NO: 9 - protein, 8 aa, Artificial Sequence
  Description of Artificial Sequence Synthetic peptide
PPKKARED

SEQ ID NO: 10 - protein, 8 aa, Artificial Sequence
  Description of Artificial Sequence Synthetic peptide
PQPKKKPL

SEQ ID NO: 11 - protein, 12 aa, Artificial Sequence
  Description of Artificial Sequence Synthetic peptide
SALIKKKKKMAP

SEQ ID NO: 12 - protein, 5 aa, Artificial Sequence
  Description of Artificial Sequence Synthetic peptide
DRLRR

SEQ ID NO: 13 - protein, 7 aa, Artificial Sequence
  Description of Artificial Sequence Synthetic peptide
PKQKKRK

SEQ ID NO: 14 - protein, 10 aa, Artificial Sequence
  Description of Artificial Sequence Synthetic peptide
RKLKKKIKKL

SEQ ID NO: 15 - protein, 10 aa, Artificial Sequence
  Description of Artificial Sequence Synthetic peptide
REKKKFLKRR

SEQ ID NO: 16 - protein, 20 aa, Artificial Sequence
  Description of Artificial Sequence Synthetic peptide
KRKGDEVDGVDEVAKKKSKK

SEQ ID NO: 17 - protein, 17 aa, Artificial Sequence
  Description of Artificial Sequence Synthetic peptide
RKCLQAGMNLEARKTKK

SEQ ID NO: 18 - protein, 9 aa, Severe acute respiratory syndrome coronavirus 2
KLFDRYFKY

SEQ ID NO: 19 - protein, 9 aa, Severe acute respiratory syndrome coronavirus 2
RQFHQKLLK